SEQUENCE LISTING (9 sequences)

SEQ ID NO: 1 - DNA, 13 bp, Artificial Sequence
  Description of Artificial Sequence Hypothetical nucleotide sequence
ctgctaacacgga

SEQ ID NO: 2 - DNA, 11 bp, Artificial Sequence
  Description of Artificial Sequence Hypothetical nucleotide sequence
tccgtgtnagn

SEQ ID NO: 3 - DNA, 20 bp, Artificial Sequence
  Description of Artificial Sequence Hypothetical nucleotide sequence
gctactgctactaacacgga

SEQ ID NO: 4 - DNA, 72 bp, Artificial Sequence
  Description of Artificial Sequence Hypothetical nucleotide sequence
aaaaaaaaaaaaaaaaaaaaaaaaaaaaaaaaaaaaaaaaaaaaaaaaaaaaaaaaaaaaaaaaaaaaaaaa

SEQ ID NO: 5 - RNA, 73 nt, Artificial Sequence
  Description of Artificial Sequence Hypothetical nucleotide sequence
uaaaaaaaaaaaaaaaaaaaaaaaaaaaaaaaaaaaaaaaaaaaaaaaaaaaaaaaaaaaaaaaaaaaaaaaa

SEQ ID NO: 6 - RNA, 21 nt, Artificial Sequence
  Description of Artificial Sequence Hypothetical nucleotide sequence
uuuuuuuuuuuuuuuuuuuuc

SEQ ID NO: 7 - DNA, 15 bp, Artificial Sequence
  Description of Artificial Sequence Hypothetical nucleotide sequence
gcgaaaaaaaaaaaa

SEQ ID NO: 8 - DNA, 10 bp, Artificial Sequence
  Description of Artificial Sequence Hypothetical nucleotide sequence
tttttttcgc

SEQ ID NO: 9 - DNA, 15 bp, Artificial Sequence
  Description of Artificial Sequence Hypothetical nucleotide sequence
aaaaaaaaaaaaaaa

(19) United States
(12) Patent Application Publication
(10) Pub. No.: US 2010/0190151 A1
(43) Pub. Date: Jul. 29, 2010

(54) FLUORESCENTLY LABELED NUCLEOSIDE TRIPHOSPHATES AND ANALOGS THEREOF FOR SEQUENCING NUCLEIC ACIDS

(76) Inventors: Stephen R. Quake, Stanford, CA (US); Christopher J. Lacenere, New York, NY (US)

Correspondence Address: MILSTEIN ZHANG & WU LLC, 49 LEXINGTON STREET, SUITE 6, NEWTON, MA, 02465-1062, US

(21) Appl. No.: 11/829,808

(22) Filed: Jul. 27, 2007

Related U.S. Application Data
(63) Continuation-in-part of application No. 10/866,388, filed on Jun. 10, 2004.
(60) Provisional application No. 60/477,426, filed on Jun. 10, 2003.
(60) Provisional application No. 60/477,429, filed on Jun. 10, 2003.
(60) Provisional application No. 60/833,747, filed on Jul. 27, 2006.

(51) Int. Cl. C12Q 1/68

(52) U.S. Cl. 435/6

(57) ABSTRACT

The invention provides methods for sequencing a nucleic acid, and particularly methods for synthesizing fluorescently labeled nucleoside triphosphates and related analogs for sequencing nucleic acids.

### RELATED APPLICATIONS

This application is a continuation-in-part of co-pending U.S. patent application Ser. No. 10/866,388, filed on Jun. 10, 2004, through which pending application this application additionally claims the benefit and priority of U.S. Provisional Application Ser. No. 60/477,426, filed Jun. 10, 2003, and U.S. Provisional Application Ser. No. 60/477,429, filed Jun. 10, 2003. This application further claims the benefit and priority of U.S. Provisional Application Ser. No. 60/833,747, filed Jul. 27, 2006. Each of the applications identified above is incorporated by reference herein in its entirety.

### FIELD OF THE INVENTION

The invention relates to methods for sequencing a nucleic acid, and more particularly, to fluorescently labeled nucleoside triphosphates and related analogs for sequencing nucleic acids.

### BACKGROUND

Completion of the human genome has paved the way for important insights into biologic structure and function. Knowledge of the human genome has given rise to inquiry into individual differences, as well as differences within an individual, as the basis for differences in biological function and dysfunction. For example, single nucleotide differences between individuals, called single nucleotide polymorphisms (SNPs), are responsible for dramatic phenotypic differences. Those differences can be outward expressions of phenotype or can involve the likelihood that an individual will get a specific disease or how that individual will respond to treatment. Moreover, subtle genomic changes have been shown to be responsible for the manifestation of genetic diseases, such as cancer. A true understanding of the complexities in either normal or abnormal function will require large amounts of specific sequence information.

An understanding of cancer also requires an understanding of genomic sequence complexity. Cancer is a disease that is rooted in heterogeneous genomic instability. Most cancers develop from a series of genomic changes, some subtle and some significant, that occur in a small subpopulation of cells. Knowledge of the sequence variations that lead to cancer will lead to an understanding of the etiology of the disease, as well as ways to treat and prevent it. An essential first step in understanding genomic complexity is the ability to perform high-resolution sequencing.

Various approaches to nucleic acid sequencing exist. One conventional way to do bulk sequencing is by chain termination and gel separation, essentially as described by Sanger et al., Proc Natl Acad Sci USA, 74(12): 5463-67 (1977). That method relies on the generation of a mixed population of nucleic acid fragments representing terminations at each base in a sequence. The fragments are then run on an electrophoretic gel and the sequence is revealed by the order of fragments in the gel. Another conventional bulk sequencing method relies on chemical degradation of nucleic acid fragments. See, Maxam et al., Proc. Natl. Acad. Sci., 74: 560-564 (1977). Finally, methods have been developed based upon sequencing by hybridization. See, e.g., Drmanac, et al., Nature Biotech., 16: 54-58 (1998).

There have been many proposals to develop new sequencing technologies based on single-molecule measurements, generally either by observing the interaction of particular proteins with DNA or by using ultra high resolution scanned probe microscopy. See, e.g., Rigler, et al., DNA-Sequencing at the Single Molecule Level, Journal of Biotechnology, 86(3): 161 (2001); Goodwin, P. M., et al., Application of Single Molecule Detection to DNA Sequencing. Nucleosides & Nucleotides, 16(5-6): 543-550 (1997); Howorka, S., et al., Sequence-Specific Detection of Individual DNA Strands using Engineered Nanopores, Nature Biotechnology, 19(7): 636-639 (2001); Meller, A., et al., Rapid Nanopore Discrimination Between Single Polynucleotide Molecules, Proceedings of the National Academy of Sciences of the United States of America, 97(3): 1079-1084 (2000); Driscoll, R. J., et al., Atomic-Scale Imaging of DNA Using Scanning Tunneling Microscopy. Nature, 346(6281): 294-296 (1990).

The high linear data density of DNA (3.4 A/base) has been an obstacle to the development of a single-molecule DNA sequencing technology. Scanned probe microscopes have not yet been able to demonstrate simultaneously the resolution and chemical specificity needed to resolve individual bases. Other proposals turn to nature for inspiration and seek to combine optical techniques with enzymes that have been fine-tuned by evolution to operate as machines that assemble and disassemble DNA with single-base resolution.

As discussed earlier, conventional nucleotide sequencing is accomplished through bulk techniques. Bulk sequencing techniques are not useful for the identification of subtle or rare nucleotide changes due to the many cloning, amplification and electrophoresis steps that complicate the process of gaining useful information regarding individual nucleotides. As such, research has evolved toward methods for rapid sequencing, such as single molecule sequencing technologies. The ability to sequence and gain information from single molecules obtained from an individual patient is the next milestone for genomic sequencing. However, effective diagnosis and management of important diseases through single molecule sequencing is impeded by lack of cost-effective tools and methods for screening individual molecules.

A need therefore exists for more effective and efficient methods for single molecule nucleic acid sequencing.

### SUMMARY OF THE INVENTION

The invention provides methods and materials for sequencing nucleic acids. In particular, the invention provides nucleotide analogs and methods of their use in nucleic acid sequencing reactions. The invention also provides methods for screening of polymerases for high density incorporation of fluorescently labeled dNTPs and synthesizing modified dNTPs.

In general terms, the invention provides a fluorescently labeled deoxynucleoside triphosphate (dNTP) and related analogs for single-molecule nucleic acid sequencing. More specifically, the invention provides a fluorescently labeled dNTP and related analogs comprising either an extended linker or a cleavable linker.

According to the invention, a fluorescently labeled dNTP and polymerase (or polymerizing agent) are added to surface-bound template nucleic acid molecules. After a wash step, a fluorescent signal is detected if there has been a successful incorporation event. This signal corresponds to individual template nucleic acid molecules that have had their primer extended by one nucleotide. After recording which template nucleic acid molecules have had a successful incorporation event, the fluorescent signal is eliminated via photo-bleaching. If no incorporation event is detected, the process is repeated with a different dNTP, and so on.

Accordingly, the invention provides parallelism and the ability to monitor hundreds of nucleic acid templates simultaneously. In a preferred embodiment, the invention makes use of fluorescence resonance energy transfer (FRET). Fluoresence resonance energy transfer is described in Weiss, S., Fluorescence Spectroscopy of Single Biomolecules, Science, 283(5408): 1676-1683 (1999); Ha, T., Single-Molecule Fluorescence Resonance Energy Transfer, Methods, 25(1): 78-86 (2001); Ha, T. J., et al., Single-Molecule Fluorescence Spectroscopy of Enzyme Conformational Dynamics and Cleavage Mechanism, Proceedings of the National Academy of Sciences of the United States of America, 96(3): 893-898 (1999); incorporated by reference herein. Using FRET, single-molecule sequence fingerprints up to five base pairs in length are obtained. The ultimate read-length is likely determined by the interaction of polymerase with the modified dNTPs and/or the modified nucleotides that have already been incorporated into the growing nucleic acid strand. dNTP analogs with extended linkers are incorporated during nucleic acid synthesis with significantly higher yields. It is also possible to use a more promiscuous polymerase to increase read-length or dNTP analogs whose dye can be removed at each step via a cleavable linker. Microfluidic integration along with automation will further complement this technology by permitting a sparing use of reagents and requiring far less time and man-power than current sequencing methodologies demand.

In general terms, the invention provides a method for nucleic acid sequence determination. According to the invention, a target nucleic acid, which is attached to a substrate, is exposed to a primer that is complementary to at least a portion of the target, a fluorescently labeled nucleoside triphosphate, and a polymerizing agent. Primer extension is conducted and the incorporation of the nucleoside in the primer is detected. Thereafter, each step is repeated to determine the sequence of the target, which can be compiled based upon the complement sequence. When detecting the incorporation of the nucleoside in the primer, coincident fluorescence emission of the first fluorescent label and the second fluorescent label is detected. The coincident fluorescence emission spectrum is between about 400 nm to about 900 nm. Coincident detection represents the presence of a single labeled molecule. The method may further include the step of washing an unincorporated nucleoside or analog thereof. In a preferred embodiment, the fluorescently labeled nucleoside triphosphate is cleaved. The cleavage step is performed by using photolysis or chemical hydrolysis.

Fluorescently-labeled nucleoside triphosphates of the invention include any nucleoside that has been modified to include a label that is directly or indirectly detectable. Such labels include optically-detectable labels such fluorescent labels, including fluorescein, rhodamine, phosphor, coumarin, polymethadine dye, fluorescent phosphoramidite, texas red, green fluorescent protein, acridine, cyanine, cyanine 5 dye, cyanine 3 dye, 5-(2′-aminoethyl)-aminonaphthalene-1-sulfonic acid (EDANS), BODIPY, ALEXA, conjugated multi-dyes, or a derivative or modification of any of the foregoing. In one embodiment of the invention, fluorescence resonance energy transfer (FRET) is employed to produce a detectable, but quenchable, label. FRET may be used in the invention by, for example, modifying the primer to include a FRET donor moiety and using nucleotides labeled with a FRET acceptor moiety. In another embodiment of the invention, the fluorescently labeled nucleoside triphosphate lacks a 3′ hydroxyl group. In a further embodiment, the fluorescently labeled nucleoside triphosphate is a non-chain terminating nucleotide. The non-chain terminating nucleotide is a deoxynucleotide selected from the group consisting of dATP, dTTP, dUTP, dCTP, and dGTP. Alternatively, the non-chain terminating nucleotide is a ribonucleotide selected from the group consisting of ATP, UTP, CTP, and GTP.

While the invention is exemplified herein with fluorescent labels, the invention is not so limited and can be practiced using nucleotides labeled with any form of detectable label, including radioactive labels, chemoluminescent labels, luminescent labels, phosphorescent labels, fluorescence polarization labels, and charge labels.

A detailed description of the certain embodiments of the invention is provided below. Other embodiments of the invention are apparent upon review of the detailed description that follows.

### BRIEF DESCRIPTION OF THE DRAWINGS

FIG. 1 is a schematic drawing of the optical setup of a conventional microscope equipped with total internal reflection (TIR) illumination;

FIG. 2 depicts DNA polymerase active on surface-anchored DNA molecules.

FIG. 3 shows the sequencing of single molecules with spFRET;

FIG. 4 shows a histogram of sequence space for 4-mers composed of A and G;

FIG. 5 shows a demonstration of “bulk” incorporation assay in the DNA sequencing chip;

FIG. 6 is an outline of the DNA polymerase screening assay;

FIG. 7 comprises the results of screening twelve thermophilic polymerases;

FIG. 8 is a schematic illustration of through-the-objective type total internal reflection (TIR) microscopy;

FIG. 9 is an example of an optics layout for multiple color excitation TIR microscopy;

FIG. 10 is a summary of directed evolution process to discover DNA polymerase mutants optimized for incorporating labeled dNTPs; and

FIG. 11 is a schematic overview of the protocol used to re-sequence the genome of E. coli.

### DETAILED DESCRIPTION OF THE DRAWINGS

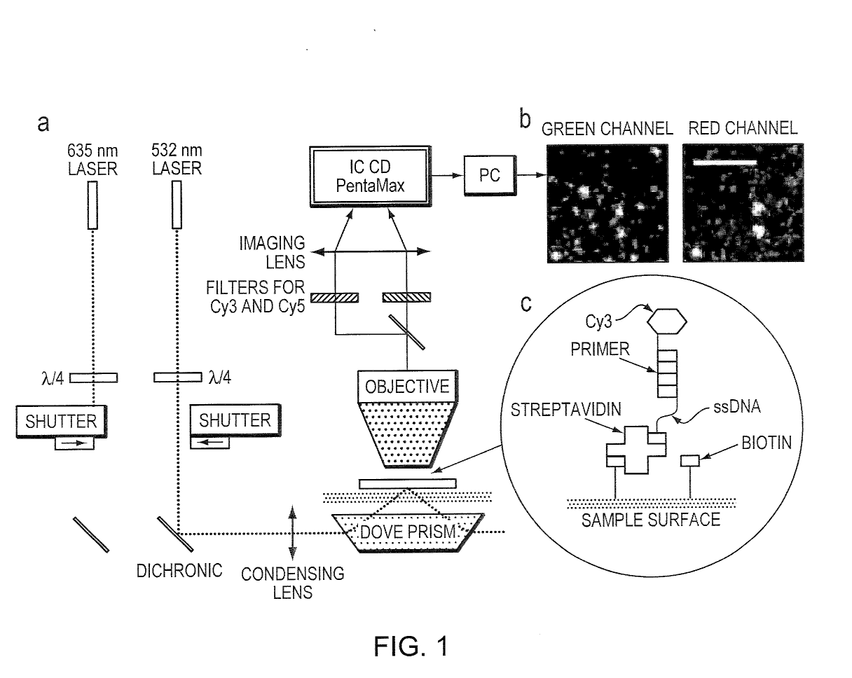

FIG. 1a is a schematic drawing of the optical setup. The green laser illuminates the surface in TIR mode while the red laser is blocked. Both Cy3 and Cy5 fluorescence spectra are recorded independently by the intensified CCD. FIG. 1b shows single-molecule images obtained by the system: Colocation of Cy3 and Cy5 labeled nucleotides with template DNA molecules being sequenced. Scale bar, 10 . mu.m. FIG. 1c is a drawing of primed template DNA molecules attached to the surface of a microscope slide via streptavidin and biotin.

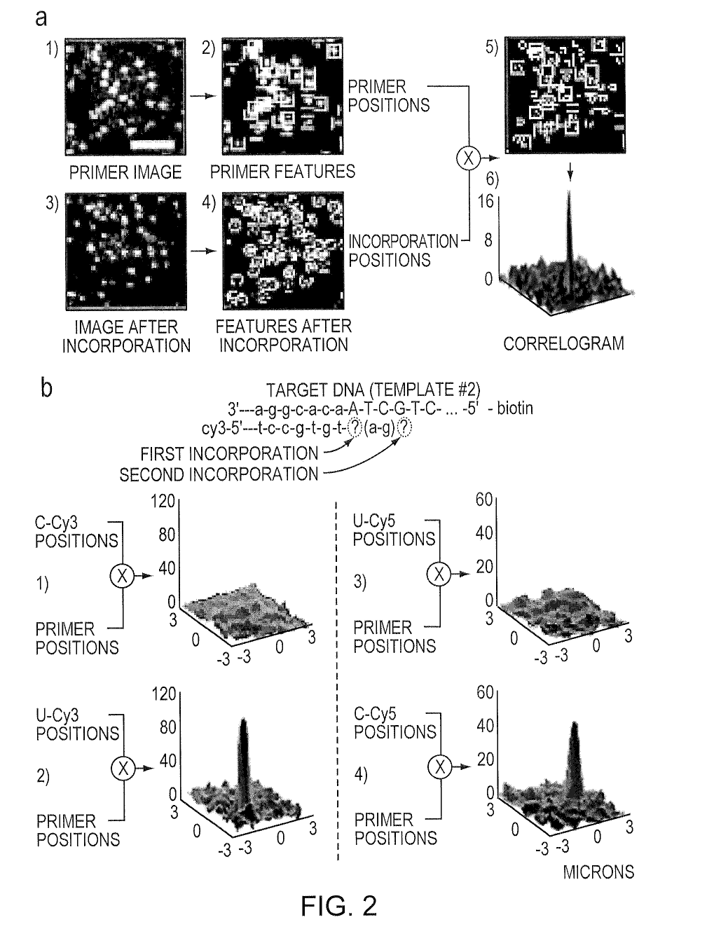

FIG. 2a shows the positional correlation of DNA template fluorescence and labeled nucleotide fluorescence due to the successful incorporation of a labeled dNTP by DNA polymerase. FIG. 2a(1) is an image of the slide surface: Annealed primer/template DNA molecules detected by Cy3 fluorescence from the labeled primer. Scale bar, 10 .mu.m. FIG. 2a(2) shows software-located positions of Cy3-labeled primer/template duplex DNA molecules on the slide surface. FIG. 2a(3) is an image of the slide surface: Labeled nucleotide fluorescence after successful incorporation by DNA polymerase. Note; prior to the incorporation reaction, the primer fluorescence shown in FIG. 2a(1) was photo-bleached. After incubation of template DNA molecules with DNA polymerase and a labeled dNTP, the sequencing chamber was flushed to prevent fluorescent detection of unincorporated labeled dNTPs. FIG. 2a(4) shows software-located positions of labeled nucleotides from (3) after a successful incorporation event. FIG. 2a(5) is an overlay of the primer/template positions with the labeled nucleotide positions. FIG. 2a(6) shows the high degree of positional correlation between primer/template fluorescence and labeled nucleotide fluorescence. FIG. 2(b) shows that DNA polymerase maintains selectivity and fidelity. FIG. 2(b)(1) depicts the polymerase correctly refusing to incorporate Cy3-dCTP. FIG. 2(b)(2) shows Cy3-dUTP correctly incorporated in the next reaction. FIG. 2(b)(3) shows DNA polymerase correctly refusing to incorporate Cy5-dUTP after extension of an unlabeled spacer region on the template DNA molecule. FIG. 2(b)(4) shows Cy5-dCTP correctly incorporated in the next reaction as detected by spFRET between Cy3-dUTP from (2) and Cy5-dCTP.

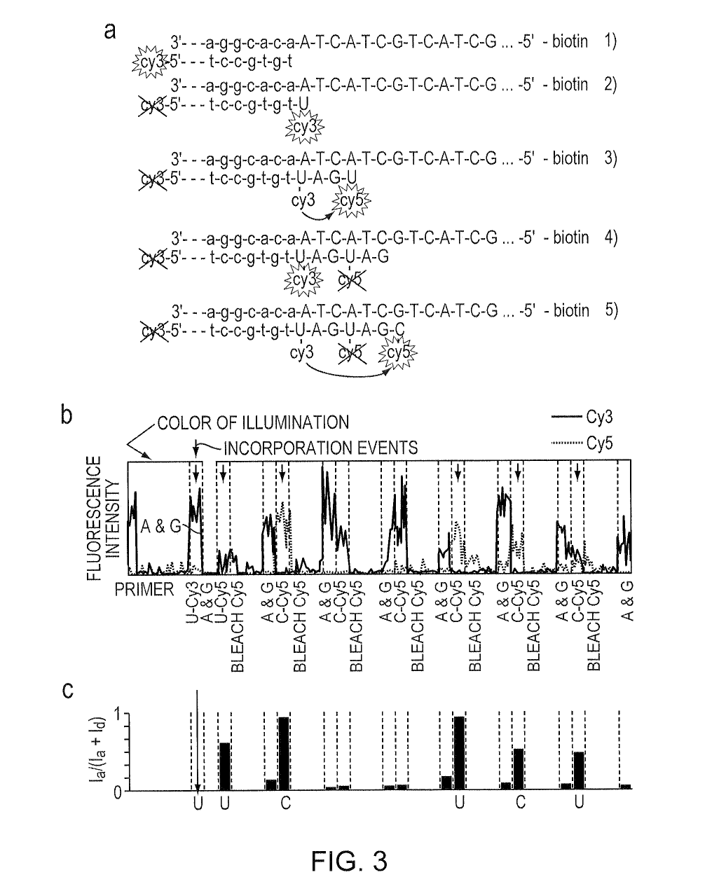

FIG. 3(a) is an illustration of the first few steps of sequencing. FIG. 3(b) shows the intensity trace from a single template DNA molecule through the entire sequencing session. The green and red lines represent the intensity of the Cy3 and Cy5 channels, respectively. Column labels indicate the last dNTP to be incubated with template DNA. Successful incorporation events are marked with an arrow. FIG. 3(c) depicts spFRET efficiency as a function of the experimental epoch to indicate successful incorporation.

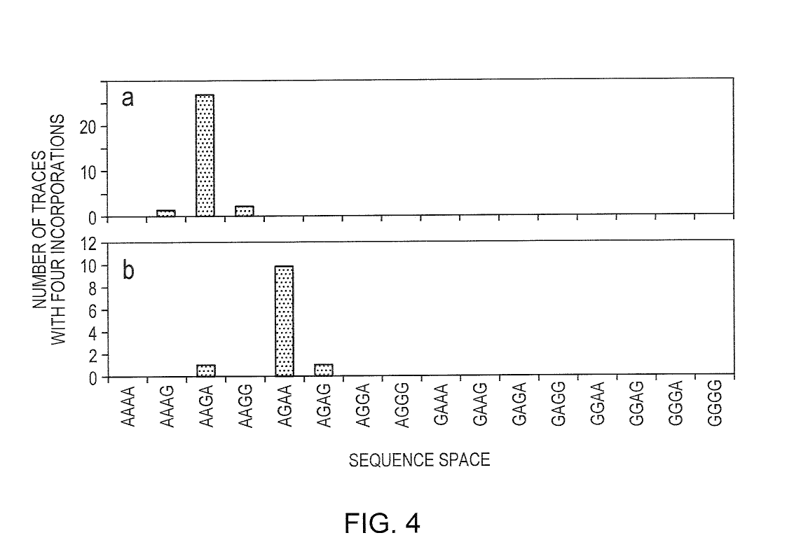

FIG. 4(a) shows the results for Template #1 (actual sequence fingerprint: AAGA). FIG. 4(b) shows the results for Template #2 (actual sequence fingerprint: AGAA). In FIG. 4, all traces that reached at least four incorporations are included.

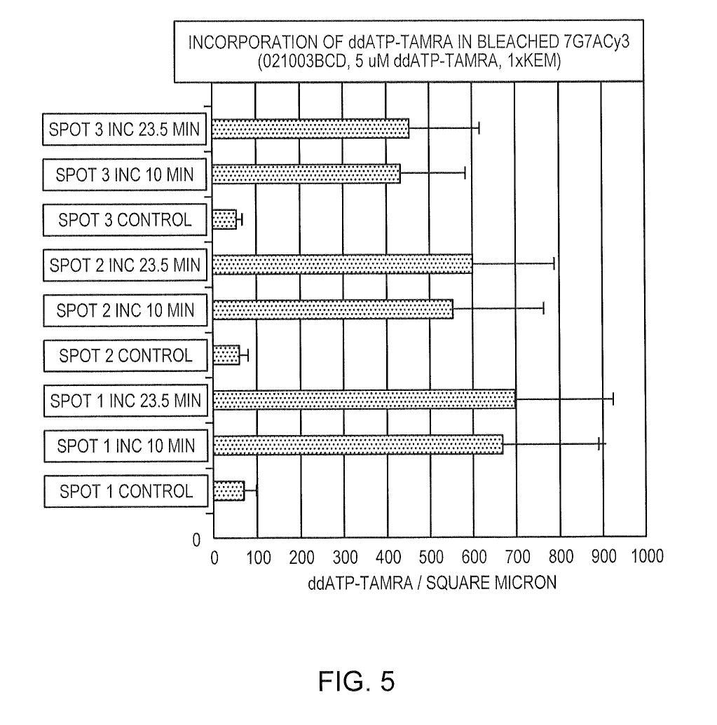

In FIG. 5, the graph shows positive fluorescent signals from fluorescently labeled ddNTP analogs as they terminate the template-dependent extension of a primer. The extending primer was first annealed to template DNA molecules and anchored to the surface of the microfluidic reaction chambers. DNA polymerase was withheld during control experiments.

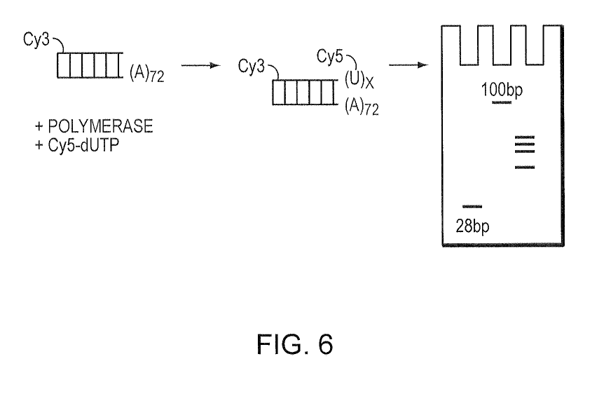

FIG. 6 depicts the extension reaction, which contains pre-annealed primer/template. Primer is conjugated to Cy3. Template DNA consists of 72 tandem A's. DNA Polymerase is then added along with Cy5-labeled dUTP to the reaction. The extension reaction is allowed to proceed for up to one hour followed by a clean-up step to remove unincorporated dNTPs. Finally, the purified reaction is run on a 10% denaturing polyacrylamide-Urea gel. Cy3 is visualized using a Typhoon 8600 Imager (Amersham Biosciences).

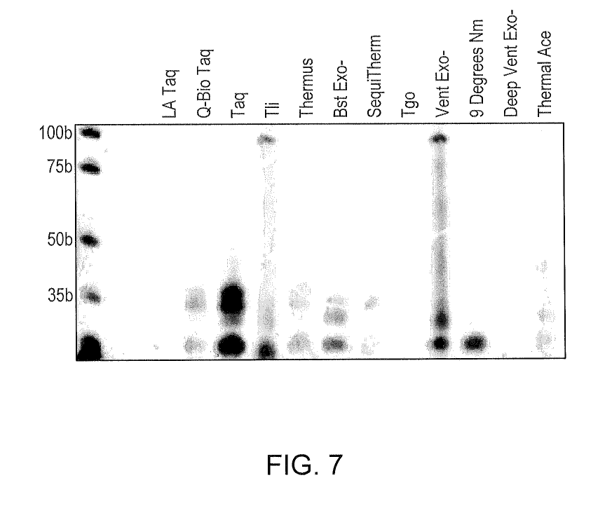

FIG. 7 depicts the results of the screening assay that demonstrates the ability of three different thermophilic DNA polymerases to incorporate up to 72 consecutive fluorescently labeled dNTPs.

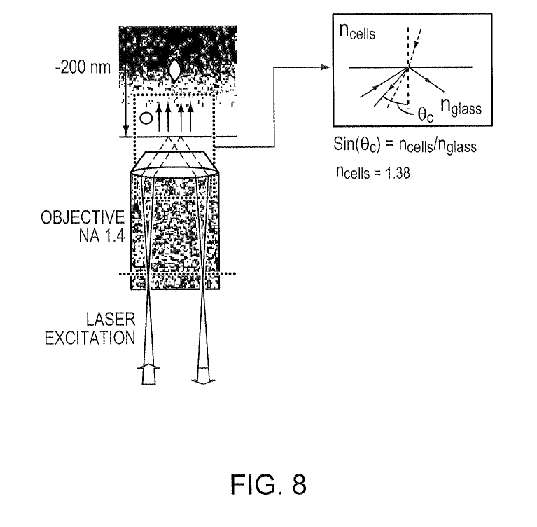

In FIG. 8, a thin layer (.about.200 nanometers) above the surface of the cover slip is illuminated by an evanescent wave, thus allowing effective excitation of fluorophores anchored near the surface while reducing background fluorescence from the solution. This depth puts an ultimate limit on the read-length for this sequencing scheme; taking into account the flexibility of the template molecule, it is calculated that this will not become a limitation on the read-length until well beyond 1,000 base pairs.

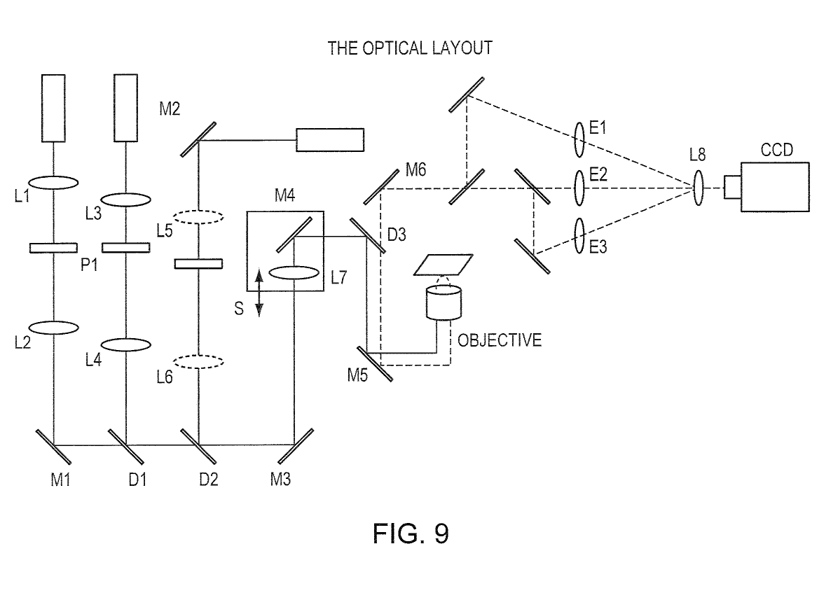

In FIG. 9, the three laser excitation is combined into a single beam with the use of polychroic mirrors, which reflect a certain wavelength range while transmitting another. A single three band polychroic is used to reflect the laser line illumination into the objective, and to pass the emissions to the imaging. The passed emissions are split into different colors again, and are cleaned with the use of emission filters.

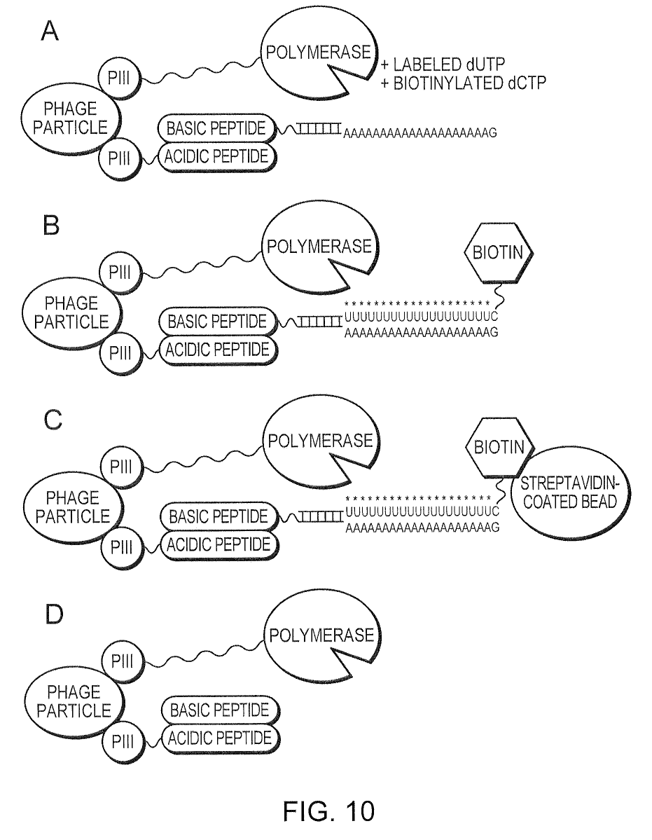

In FIG. 10A, mutant DNA polymerase library is fused to the minor phage coat protein pill. An acidic leucine zipper peptide, also fused to pIII, is used to couple a template DNA strand to the phage particle. Recombinant phage faithfully display both the pIII:polymerase and pIII:acidic leucine zipper protein fusions. Mutations are introduced into DNA polymerase using 2-step overlapping extension PCR. The selective template DNA molecule contains a basic leucine zipper fused to a stretch of 20 A's followed by a single G. This basic leucine zipper binds the acidic leucine zipper with high affinity. In FIG. 10B, phage particles displaying both a mutant DNA polymerase as well as the selective DNA template are incubated with dye-labeled dUTP and biotinylated dCTP. In FIG. 10C, after the extension reaction is carried to completion, streptavidin coated beads are added to the mix. These beads bind biotin and allow phage particles displaying completely extended template to be spun down. In FIG. 10D, after centrifugation of the streptavidin coated beads, DNAse is added to the spun-down phage particles, causing them to be released and characterized further. These phage particle candidates potentially produce mutant DNA polymerases capable of incorporating successive, labeled dNTPs.

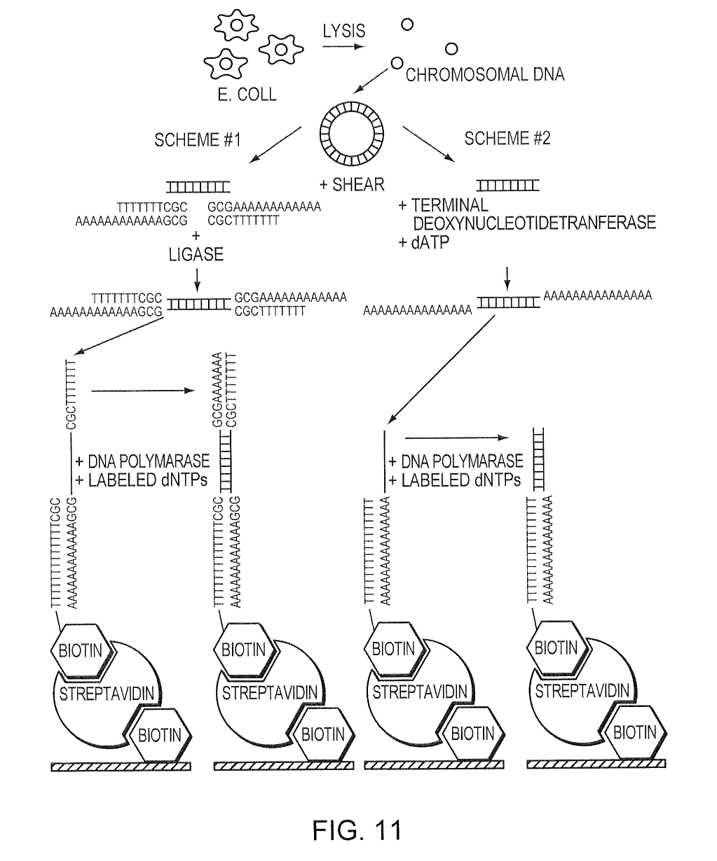

FIG. 11 shows the genomic DNA sample preparation scheme for re-sequencing the E. coli genome.

### DETAILED DESCRIPTION OF THE INVENTION

The invention provides methods for sequencing single molecules of nucleic acids. A nucleic acid can come from a variety of sources. For example, nucleic acids can be naturally occurring DNA or RNA isolated from any source, recombinant molecules, cDNA, or synthetic analogs, as known in the art. For example, a nucleic acid may be genomic DNA, genes, gene fragments, exons, introns, regulatory elements (such as promoters, enhancers, initiation and termination regions, expression regulatory factors, expression controls, and other control regions), DNA comprising one or more single-nucleotide polymorphisms (SNPs), allelic variants, and other mutations. Also included is the full genome of one or more cells, for example cells from different stages of diseases such as cancer. The nucleic acid may also be mRNA, tRNA, rRNA, ribozymes, splice variants, antisense RNA, and RNAi. Also contemplated according to the invention are RNA with a recognition site for binding a polymerase, transcripts of a single cell, organelle or microorganism, and all or portions of RNA complements of one or more cells, for example, cells from different stages of development or differentiation, and cells from different species.

Nucleic acids can be obtained from any cell of a person, animal, plant, bacteria, or virus, including pathogenic microbes or other cellular organisms. Individual nucleic acids can be isolated for analysis. Nucleic acids may be obtained from a variety of biological samples, such as blood, urine, cerebrospinal fluid, seminal fluid, saliva, breast nipple aspirate, sputum, stool and biopsy tissue. Especially preferred are samples of luminal fluid because such samples are generally free of intact, healthy cells. However, any tissue or body fluid specimen may be used according to methods of the invention.

Observations of single-molecule fluorescence can be made using a conventional microscope equipped with total internal reflection (TIR) illumination. TIR microscopy illuminates a planar field approximately 200 nm above the slide surface, thus significantly reducing background fluorescence (FIG. 1). First, the surface of a quartz slide is chemically treated to specifically anchor template nucleic acid molecules while preventing non-specific binding of labeled dNTPs present in the sequencing reaction. A plastic flow cell is then attached to the surface of the slide to facilitate the exchange of buffers and reagents. Next, biotinylated oligonucleotides, serving as sequencing templates, are annealed to a fluorescently labeled primer. Template nucleic acid molecules are bound to the slide surface via streptavidin and biotin at a surface density low enough to resolve single nucleic acid molecules. The primed templates are detected via their fluorescent tags and their locations are recorded for future reference. After noting the location of each template nucleic acid molecule, their fluorescent tags are photo-bleached. Labeled dNTPs and polymerase (or polymerizing agent) are then washed in and out of the flow cell, one dNTP at a time, while the known locations of the template nucleic acid molecules are monitored for fluorescence, an indication that the primer annealed to the template nucleic acid molecule had been extended by one labeled nucleotide. With this technique, it is shown that polymerase is active on surface-immobilized nucleic acid molecules and that it can incorporate dNTPs and dye-labeled dNTP analogs with high fidelity (FIG. 2).

A confounding factor in previous attempts to sequence single nucleic acid molecules with fluorescence microscopy has been an inability to control background fluorescence and fluorescent impurities. In one embodiment, the present invention uses a combination of evanescent wave microscopy and spFRET to reject unwanted background noise (FIG. 3). The donor fluorophore used during spFRET can excite acceptor molecules only if they are within the Forster radius. The Forster radius for the Cy3 and Cy5 fluorophores used in the present invention is about 5 nm (ca. 15 bp), effectively creating an extremely high-resolution near-field source. The spatial resolution of this method exceeds the diffraction limit of conventional near-field microscopy by an order of magnitude and conventional far-field microscopy by a factor of 50. Using spFRET, single-molecule sequence fingerprints up to five base pairs in length can be obtained.

As shown in the series of experiments in FIG. 4, the unique sequence of two different template DNA molecules was correctly identified. Such single molecule sensitivity can be used to sequence millions of molecules in a massively parallel fashion.

The graph in FIG. 5 shows the signal commonly observed while “bulk” DNA sequencing on microfluidic chips, in this case using a rhodamine-labeled ddNTP analog. These experiments do not have single-molecule sensitivity, but observe fluorescence from a population of identical template DNA molecules using epifluorescence microscopy. The negative control contains no DNA polymerase. The presence of DNA polymerase results in a much stronger signal due to the successful incorporation of labeled ddNTPs into the template-dependent growing DNA strand. In this “bulk” sequencing experiment, primer extension is terminated by the ddNTPs after being incorporated. Spots refer to individual sequencing chambers. Increasing the number of polyelectrolyte layers may decrease non-specific binding and background noise even further.

The present invention is further directed to increasing sequence read-length by screening polymerases for an improved ability to incorporate successive, labeled dNTPs into the template-dependent extension of a primer. A description of the DNA polymerase screening assay is outlined in FIG. 6. FIG. 6 depicts the attempt to extend a labeled primer by as many as 72 consecutive fluorescently labeled nucleotides (e.g. Cy5-dUTP) using a poly(A) DNA template. The length of primer extension is determined by sizing the single-stranded primer strand on a denaturing polyacrylamide gel. An un-extended primer will run at 28 by (FIG. 6, lane 1), a completely extended primer will run at 100 by (FIG. 6, lane 2), and a partially extended primer will run between 100 by and 28 by (FIG. 6, lane 3). Because Cy3 is visualized, which is only present on the primer, potential problems associated with quenching of the densely Cy5-labeled extension product are avoided.

The results of screening twelve thermophilic polymerases are shown in FIG. 7. In this assay, Klenow fragment performs comparably to Taq. This assay successfully identified conditions for several candidate polymerases including Tli, Vent Exo-, and Invitrogen's ThermalAce. These results indicate that read-lengths greater than 72 base pairs are possible using commercially available polymerases and dNTP analogs.

Still a further aspect of the present invention is directed to methods for synthesizing fluorescently labeled nucleoside triphosphates and related analogs for single-molecule DNA sequencing.

Cy5-dUTP (1) is a commercially available labeled dNTP that can be used successfully to sequence DNA at the single-molecule level.

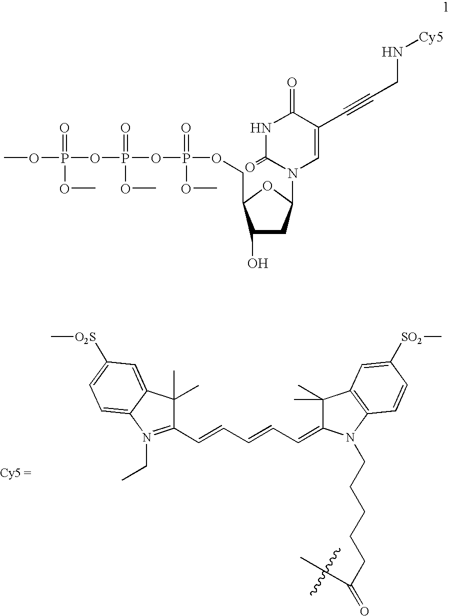

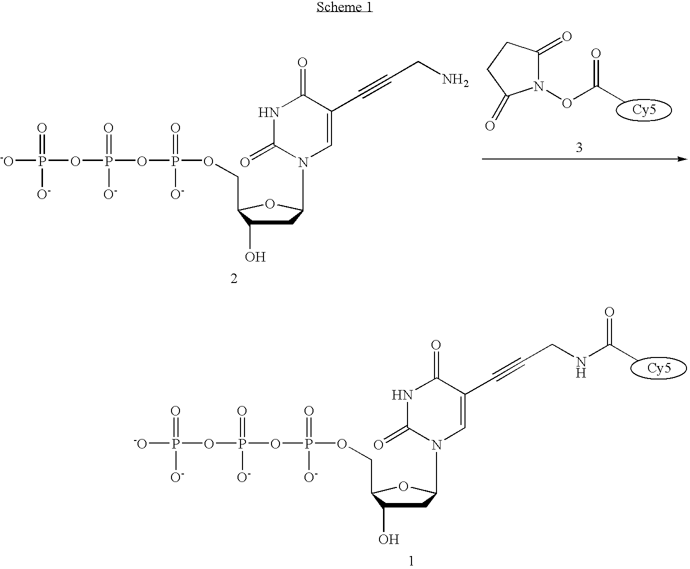

Compound 1 is synthesized via the coupling of known propargyl amine 2 and the commercially available succinimidyl ester derivative of Cy5 (3, Scheme 1). These same building blocks, 2 and 3, are used to prepare a variety of modified dNTPs that are not currently commercially available. Although Cy5 (3) is shown in this example, this invention includes any fluorescent molecules.

Aminopropynyl-dUTP 2 is easily coupled to a free acid or a succinimidyl ester derivative of the fluorophores of interest.

Modified nucleoside triphosphates containing alternative 5-position connectors can be synthesized for single-molecule DNA sequencing and are shown below (4-8). Although uridine is shown as an example, all deoxynucleosides (A,T,U,C,G) are included in this invention.

The ability of DNA polymerase to incorporate fluorescently labeled dNTPs may be reduced as a result of the increased steric bulk of the dye molecule. This directly affects the read-length of this sequencing mechanism. In order to address this issue, fluorescently labeled dNTPs that contain either an extended linker or a cleavable linker have been synthesized.

As shown below, fluorescently labeled nucleoside triphosphates containing extended linker arms have been synthesized for single-molecule DNA sequencing (9). Although uridine is shown as an example, all deoxynucleosides (A,U,C,G) are included in this invention. Also, although the example shows a derivative of 2, derivatives of 4 are also included in this invention. The extended linkers are generally composed of a carboxyl acid functionality and a heteroatom. In the formula of the extended linker below, n is an integer from 1 to about 20, preferably from about 5 to about 15, more preferably from about 5 to about 10, most preferably about 6, and m is an integer from 1 to about 20. Any chemical chain can link these two functional groups.

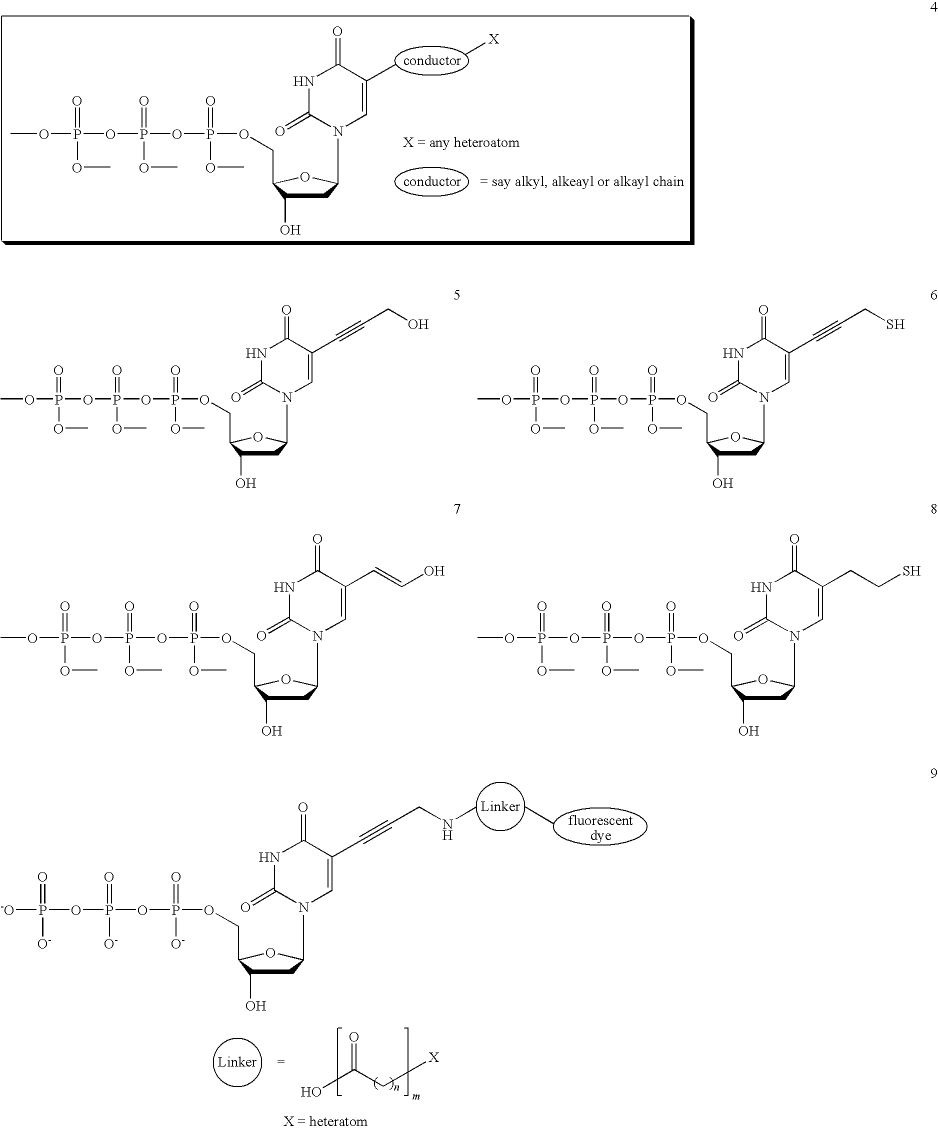

Linkers of varying length can be prepared using standard peptide synthesis techniques with any amino acid building blocks. For example, commercially available 6-aminohexanoic acid (10, Scheme 2) is extremely useful as a linker itself, capable of extending the chain between dNTP and fluorophore by seven atoms.

For example, Scheme 2 illustrates the synthesis of a dUTP fluorescent dye conjugate with a 28-atom linker (11), prepared by two simple amide bond forming reactions. It is anticipated that these long, aliphatic linkers may exhibit limited solubility in aqueous media. Ethylene glycol amino acid derivative 12 can be used in place of the aliphatic linkers 10 to increase the solubility of compounds in aqueous solution.

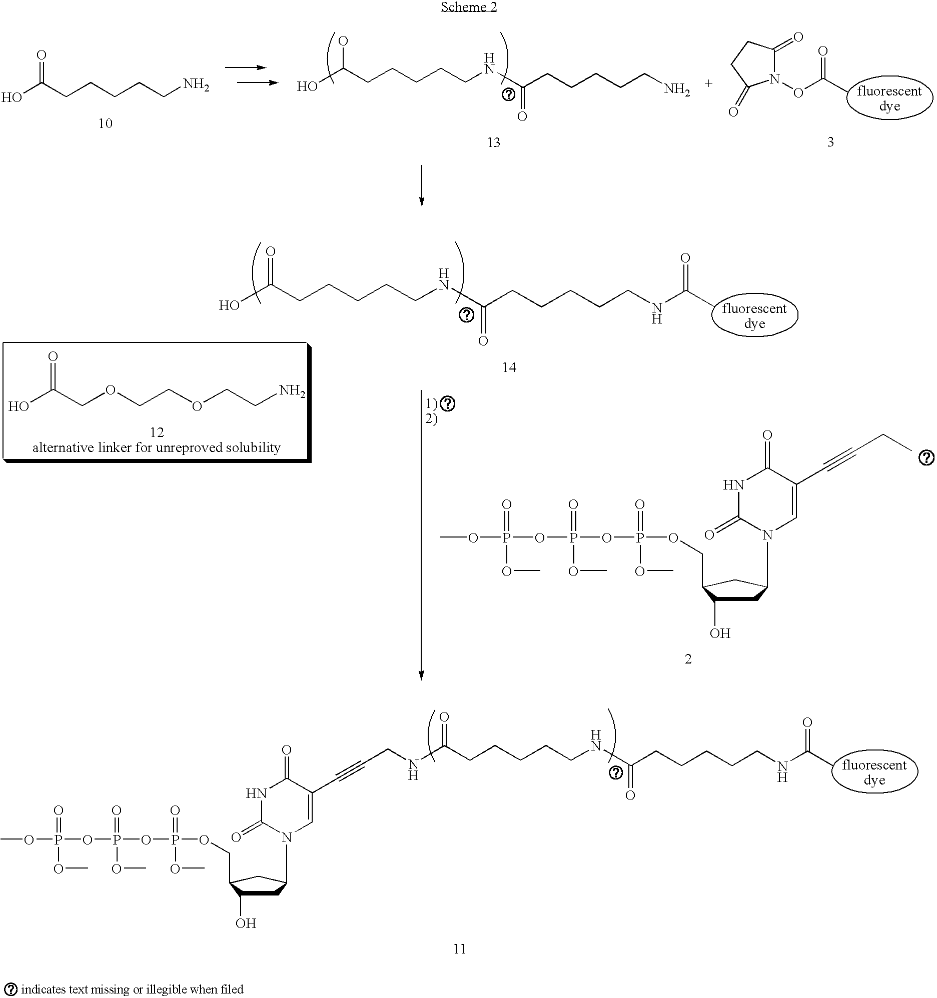

While extended linkers have proven to be a valuable approach for decreasing steric congestion along the growing strand of DNA, an alternate strategy uses a removable linker and dye (15, Scheme 3). In this scenario, once the modified dNTP is incorporated onto the growing DNA chain, the fluorophore and linker can be removed by a photo-induced or chemically triggered cleavage. Once the bulky fluorophore is removed, it is anticipated that a less sterically encumbered system will result and, therefore, higher polymerase efficiency. Although uridine is shown as an example, all deoxynucleosides (A,U,C,G) are included in this invention. Also, although the example shows a derivative of 2, compounds of derivatives of 4 are also included in this invention.

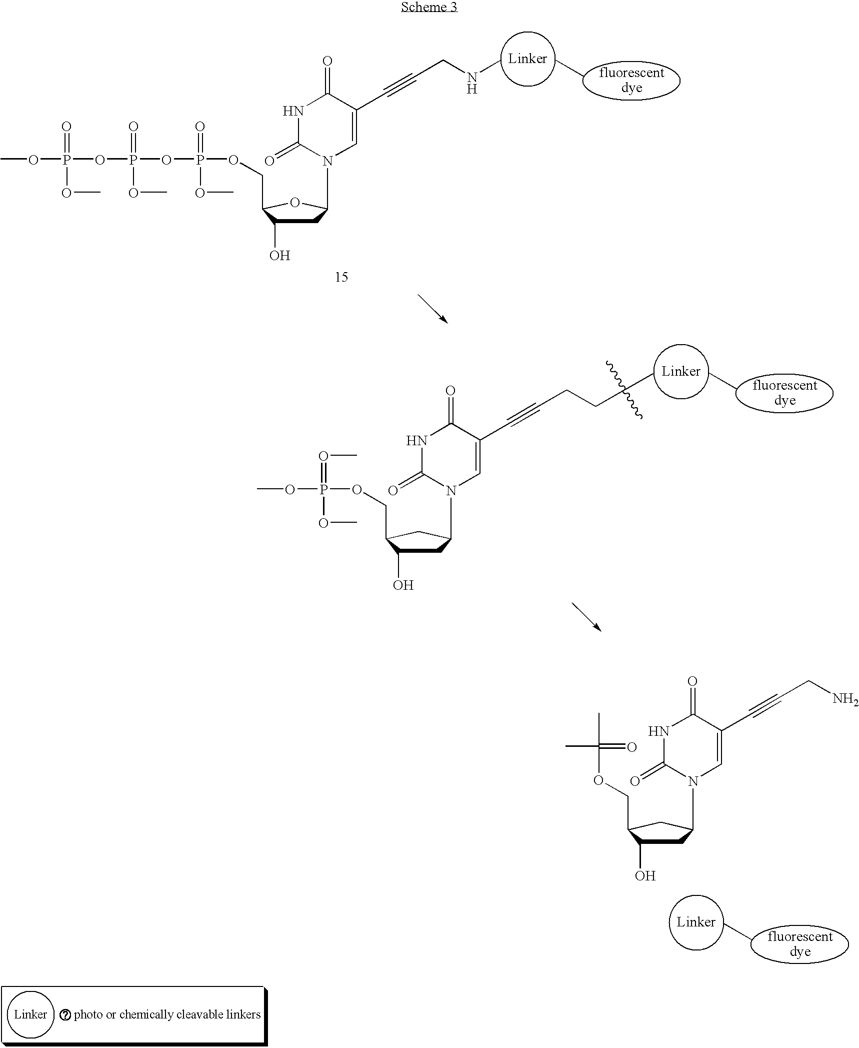

DNA polymerase can incorporate a modified dNTP containing a 2-nitrobenzyl linker that bridges a dNTP and a fluorophore, which can be removed by photolysis at 340 nm. As a result, the synthesis of such fragments in single-molecule DNA sequencing will provide a variety of dNTP-fluorophore conjugates. A host of such molecules, for example, is envisioned below (16-19):

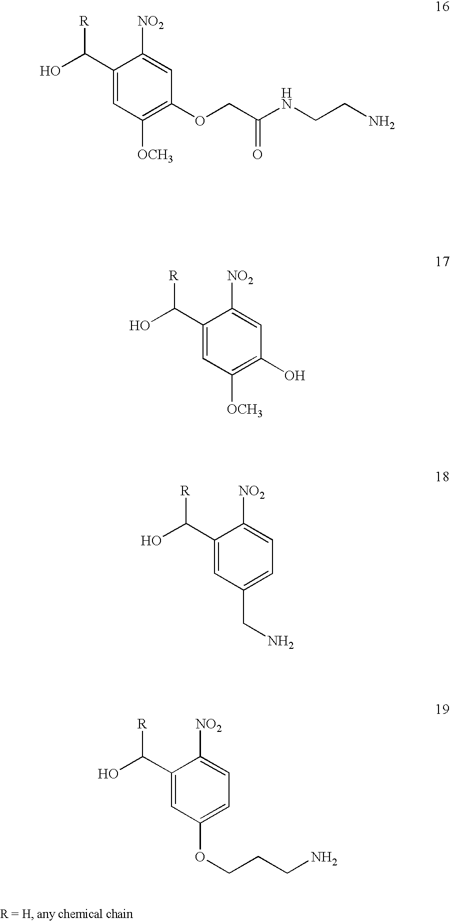

For example, linker 16 can be synthesized from known acid 20 through a DCC-mediated coupling with ethylene diamine, followed by reduction of the ketone functionality (Scheme 4). Amino alcohol 16 can then be converted to photocleavable labeled dNTP 21, via two successive peptide bond forming reactions.

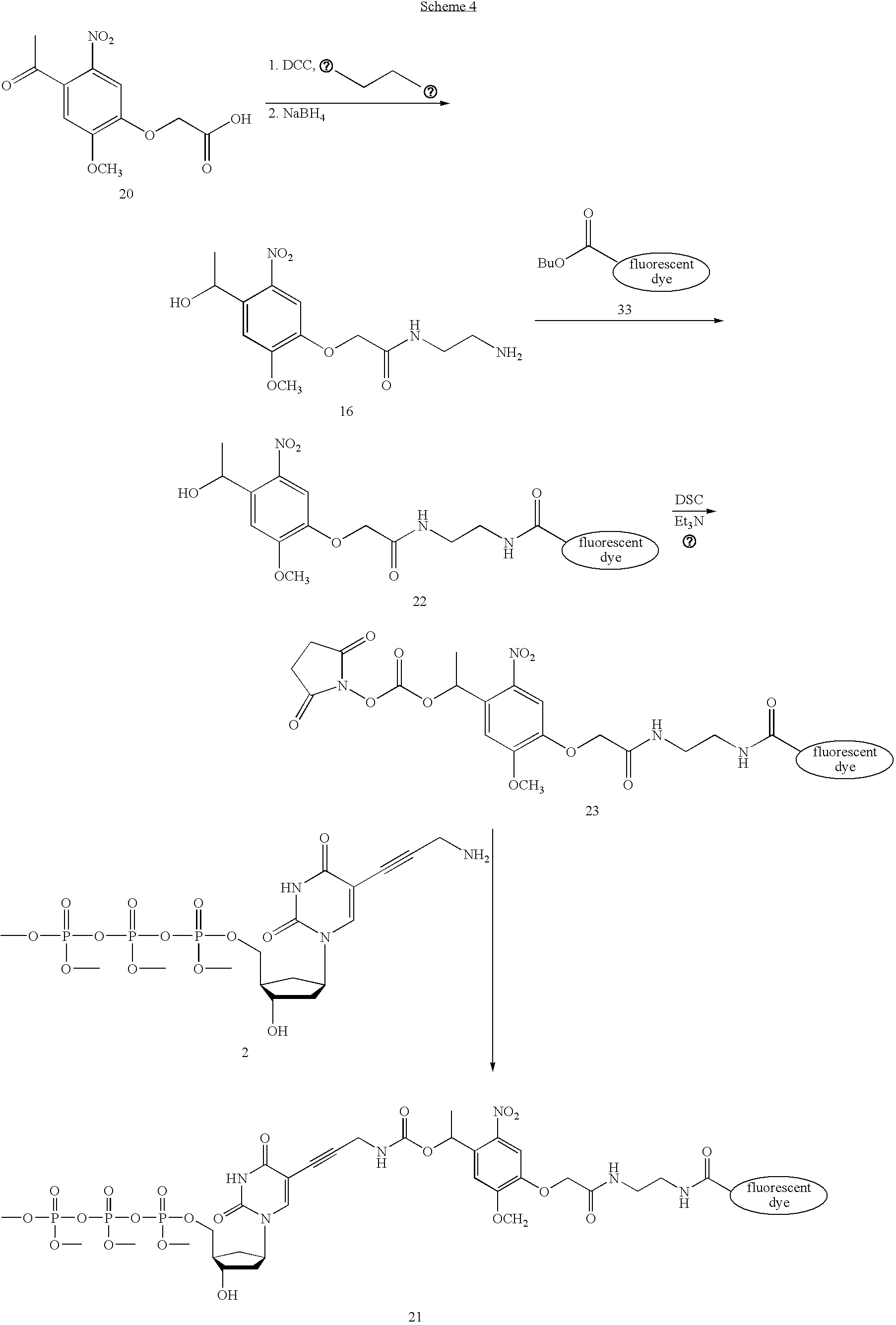

An alternative strategy for linkers involves those that are cleaved chemically rather than photolytically. Amino acid and hydroxy acid derivatives are especially appealing since they will allow for the rapid synthesis of multiple dNTP derivatives through simple amide and ester bond forming reactions. However, this invention is not limited to amino acid and hydroxy acid derivatives. Any chemical removable linker is included in this invention.

Specific conditions are required for each linker to induce cleavage. Chemically cleavable linkers can be cleaved under acidic, basic, oxidative, or reductive conditions. For example, amino acid 24 or commercially available alcohol 25 can be linked to a fluorophore and then cleaved by either base or enzyme-promoted hydrolysis of the ester bond. Another base-labile linker is 26, which has similar reactivity to the FMOC (fluorenylmethoxycarbonyl) protecting group. Amino acid linkers 27 and 28 will allow for dye removal under acidic conditions as the acetal moieties can be gently hydrolyzed. Alternatively, .alpha.-substituted pentenoic acid derivative 29 will promote the liberation of the fluorophore under oxidative iodolactonization conditions, while the disulfide functionality within 30 will provide a substrate suitable for reductive cleavage. Finally, linker diene 31 will allow for release of the fluorophore under aqueous ring closing metathesis conditions.

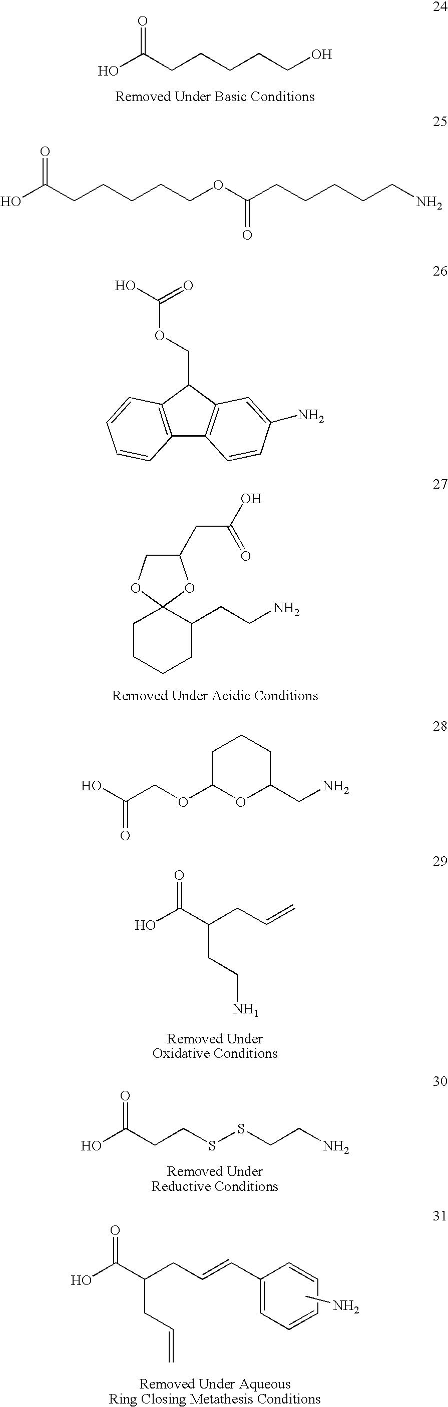

Scheme 5 below illustrates the synthesis of a modified dNTP containing a base-sensitive linker unit 35. Known FMOC amino alcohol 32 is coupled to fluorescent succinimidyl ester 33, then treated with disuccinimidyl carbonate to produce 34. Activated fluorescent FMOC derivative 34 is then linked to aminopropynyl dUTP 2 to yield the desired chemically labile dNTP 35.

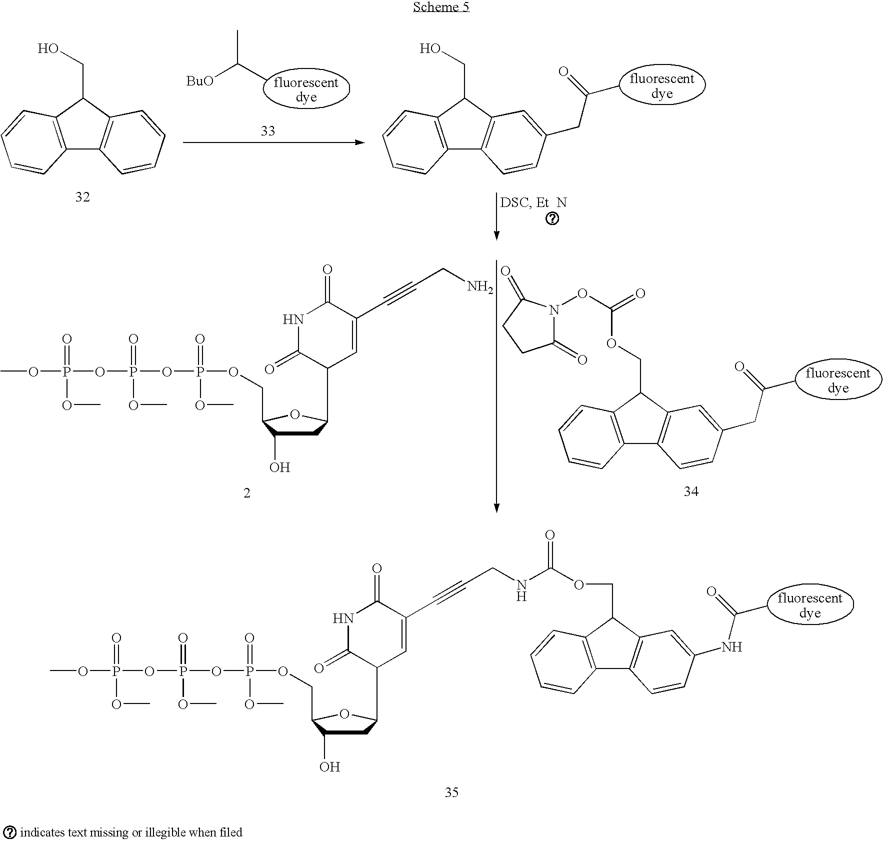

An alternative to modifying the linker region of 2 is to relocate the dye to the 3′ hydroxyl position of the ribose ring with a removable linker. Such a nucleotide would have the added benefit of halting DNA synthesis after each incorporation event until the 3′ linker is removed, whereupon the reactive alcohol will be exposed (36, Scheme 6). A major advantage of protecting the 3′ sugar carbon is that all four dNTPs may be added to the sequencing reaction at once, each labeled with a different colored dye. This should theoretically increase throughput four-fold as well as increase the accuracy at which nucleotide repeats are read. Although uridine is shown as an example, all deoxynucleosides (A,T,U,C,G) are included in this invention. Also, although the example shows a derivative of 2, compounds of derivatives of 4 are also included in this invention.

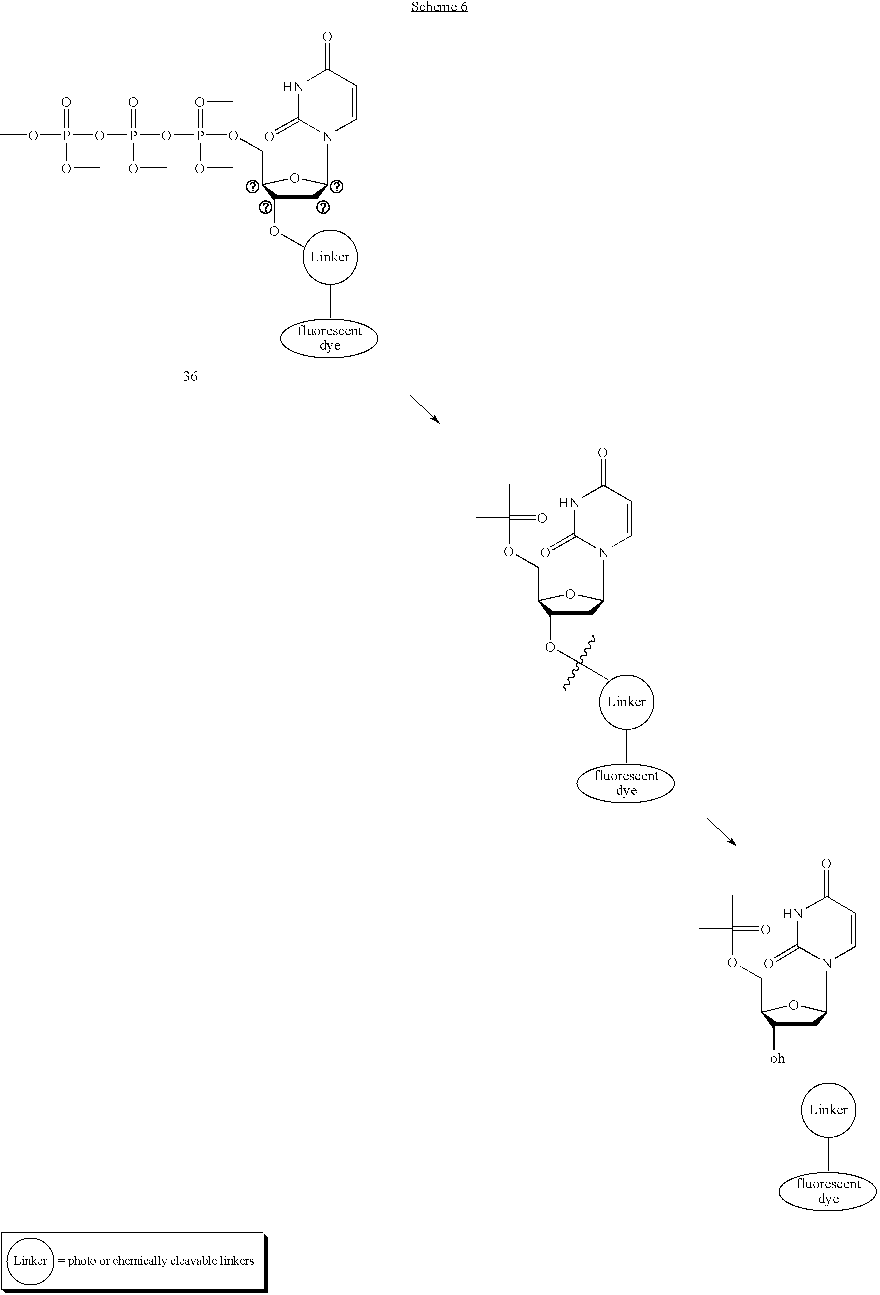

Photocleavable linkers can be used as 3′ modified dNTPs, using derivatives of the 2-nitrobenzyl linkers shown above. For example, Scheme 7 illustrates the synthesis of compounds with 3′ photoremovable linkers. The 3′ hydroxyl of commercially available deoxyuridine 37 can be alkylated with bromide 38 following initial silyl protection of the 5′ alcohol. Acid-mediated cleavage of the silyl ether will release the 5′ free hydroxyl 39 and triphosphorylation by the method described in Ludwig, J. et al., Rapid and Efficient Synthesis of Nucleoside 5′-O-(1-thiotriphosphates), 5′-triphosphates and 2,3′-cyclophosphorothioates using 2-chloro-4H-1,3,2-benzodioxaphosphorin-4-one, Journal of Organic Chemistry, 54(3): 631-635 (1989) will yield nucleoside triphosphate 40 as the free amine. Finally, 40 can be coupled to fluorophore 33 furnishing the 3′ modified nucleoside triphosphate 41.

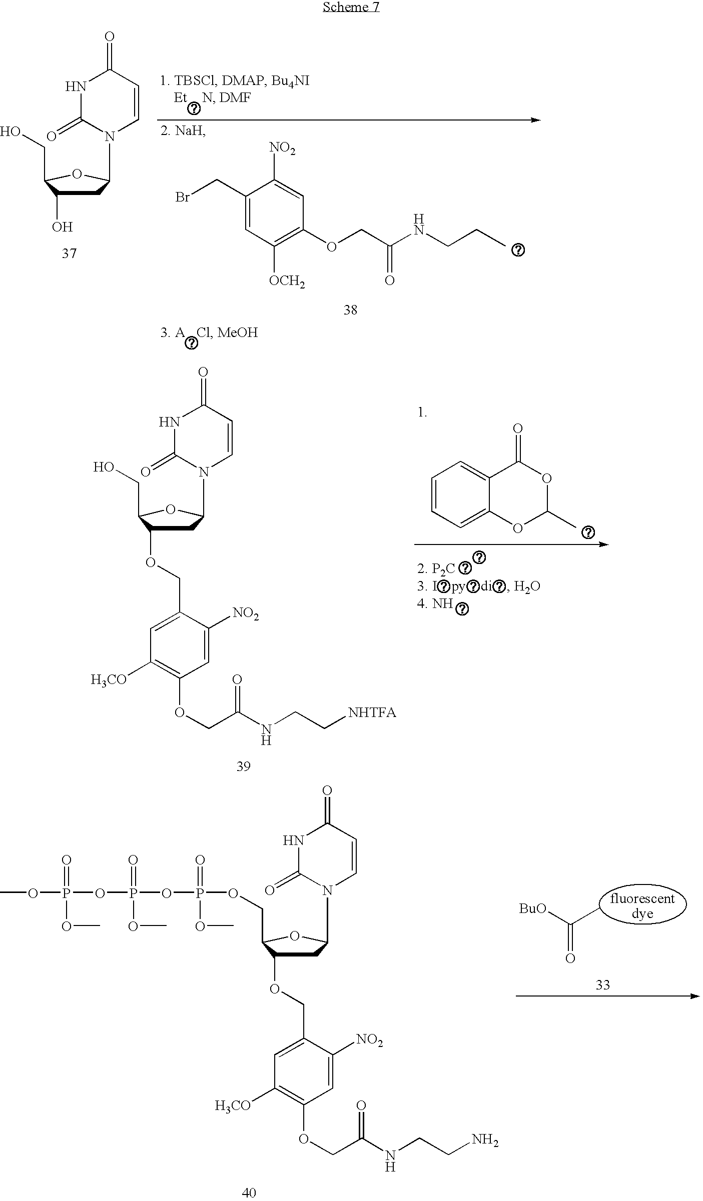

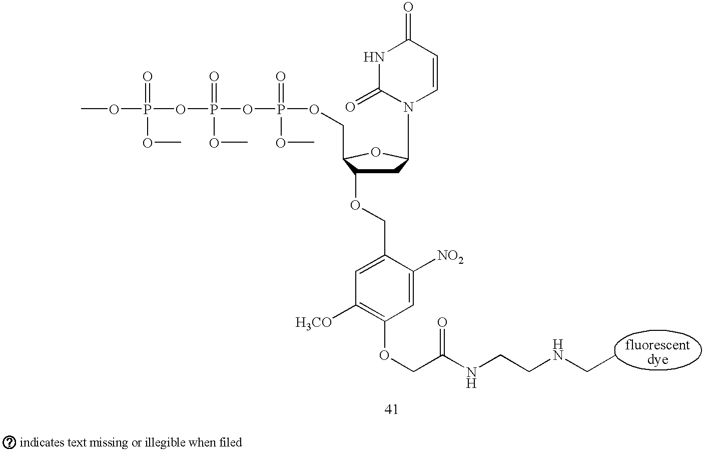

The chemical-promoted cleavage of fluorophores stemming from the 3′ sugar position is also a viable option that offers the benefits of controlled chain termination. Such a synthetic dNTP will contain a fluorophore stemming from the 3′ hydroxyl via an ester linkage. After incorporation of the dNTPs of this type by DNA polymerases, either a mild chemical cleavage of the fluorophore via base-promoted hydrolysis or an enzymatic cleavage to liberate the 3′ hydroxyl group will occur.

It has been reported in some cases that DNA polymerases are not tolerant of bulky linkers stemming from the 3′ position of the dNTP sugar. An alternative chain terminating dNTP containing only a removable protecting group on the 3′ hydroxyl group of the sugar and a fluorescent dye on the base (via a photo- or chemically cleavable linker as described above) can be synthesized for single-molecule DNA sequencing (42, Scheme 8). Once cleavage is triggered, the 3′ protecting group as well as the fluorescent dye will be released simultaneously. Although uridine is shown as an example, all deoxynucleosides (A,U,C,G) are included in this invention. Also, although the example shows a derivative of 2, compounds of derivatives of 4 are also included in this invention.

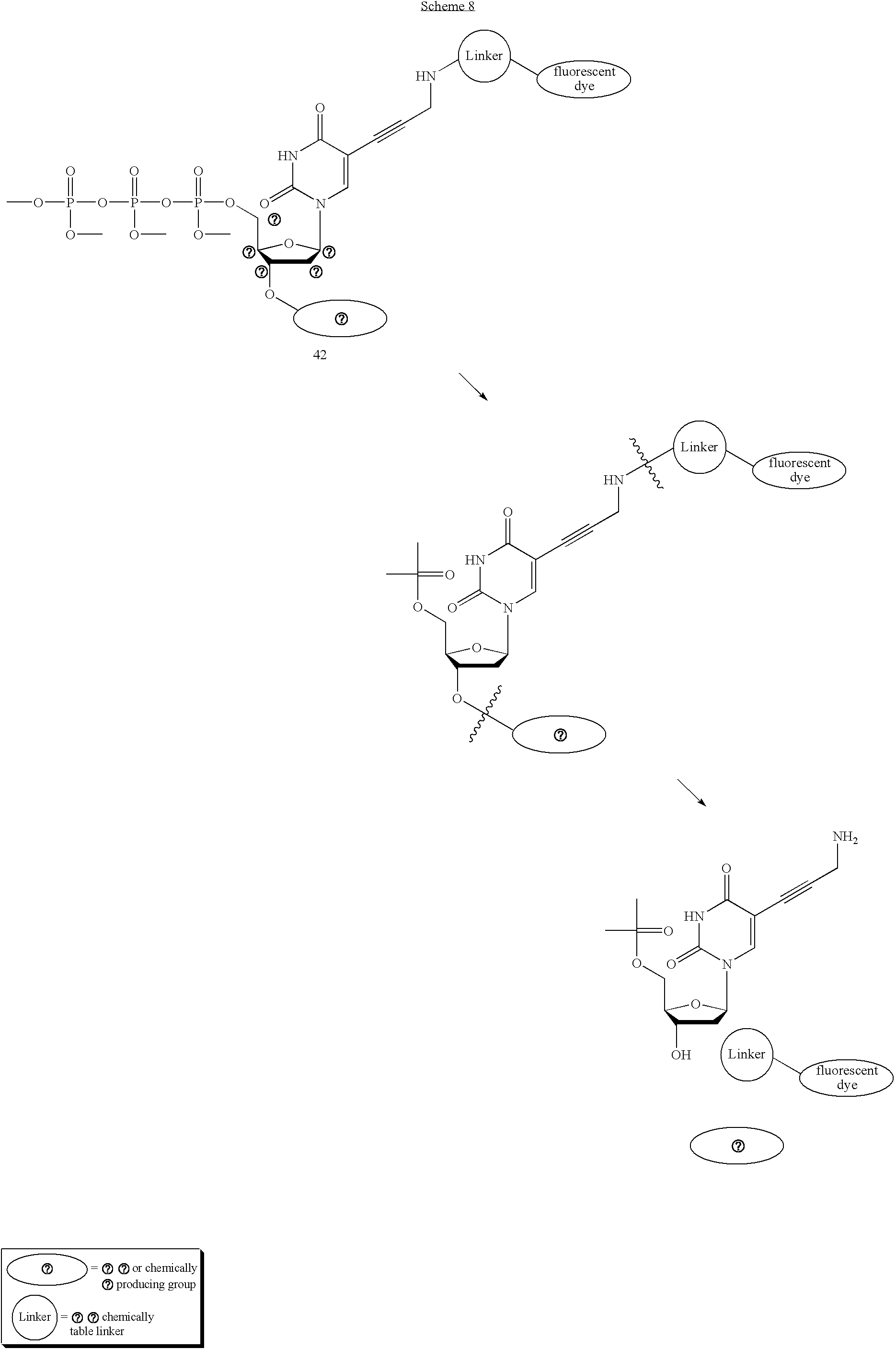

For example, hybrid 43 can be prepared from a combination of synthetic methods previously described (Scheme 9). Protection of the 3′ hydroxyl with commercially available benzylic bromide 45 can be accomplished with the aid of protecting group manipulations at the 5′ position to afford 46. 46 can then be triphosphorylated by the method of Ludwig et al. described above to yield free amine 47. This will undergo facile amide bond formation when treated with succinimidyl ester 23 (see Scheme 4 for synthesis of 23) to produce the modified dNTP 43, which contains both a removable 3′ protecting group and a removable dye attached through a photocleavable linker arm.

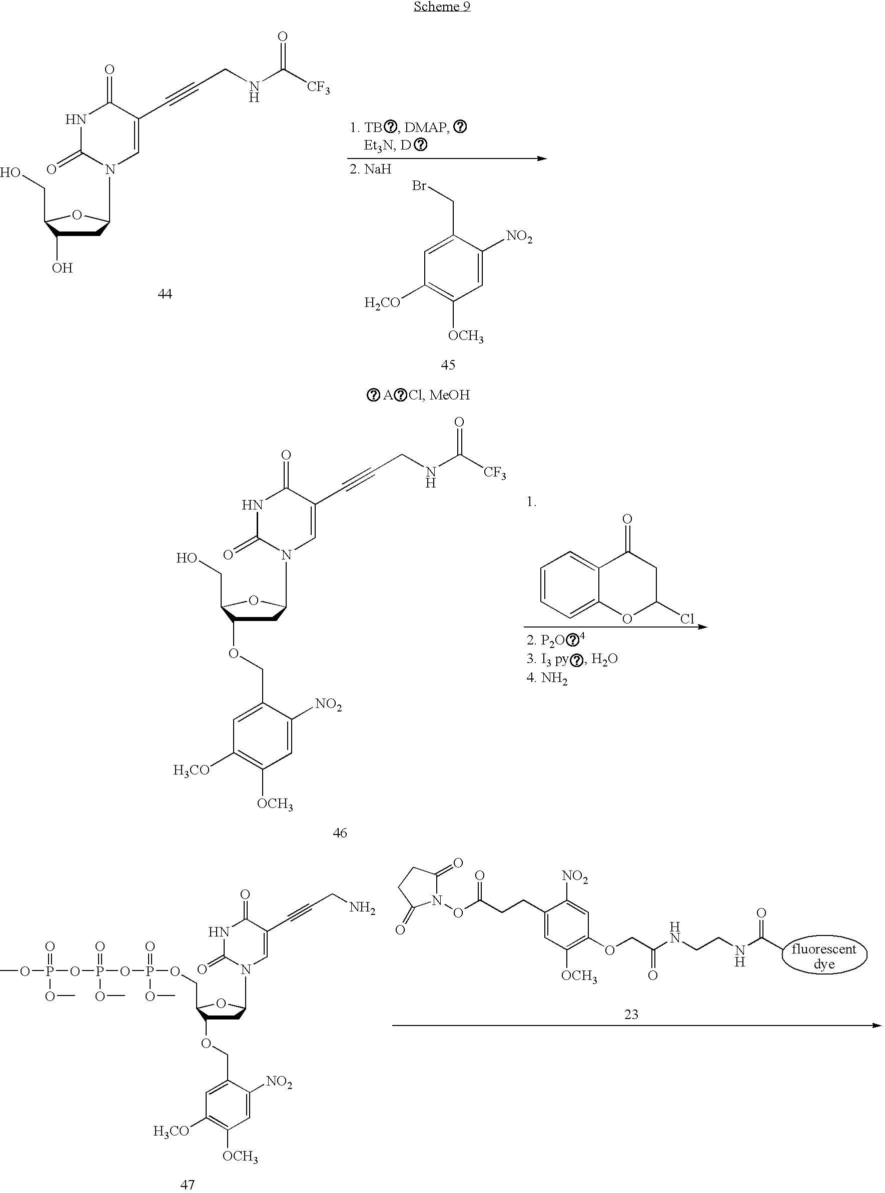

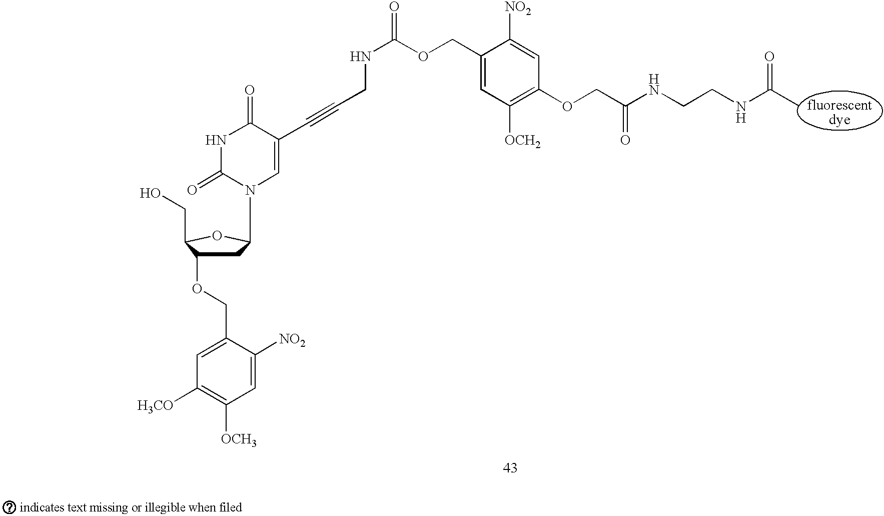

An alternative strategy is a fluorescently labeled, chain terminating, dNTPs containing both a masked (rather than protected) 3′ hydroxyl and a fluorescent dye on the base (via a photo- or chemically cleavable linker as described above) for single-molecule DNA sequencing (48, Scheme 10). After incorporation, the 3′ hydroxyl will be unveiled and the fluorophore will be cleaved, thus allowing for subsequent incorporation events. Although deoxyuridine is shown as an example, all deoxynucleosides (A,U,C,G) are included in this invention. Also, although the example shows a derivative of 2, compounds of derivatives of 4 are also included in this invention.

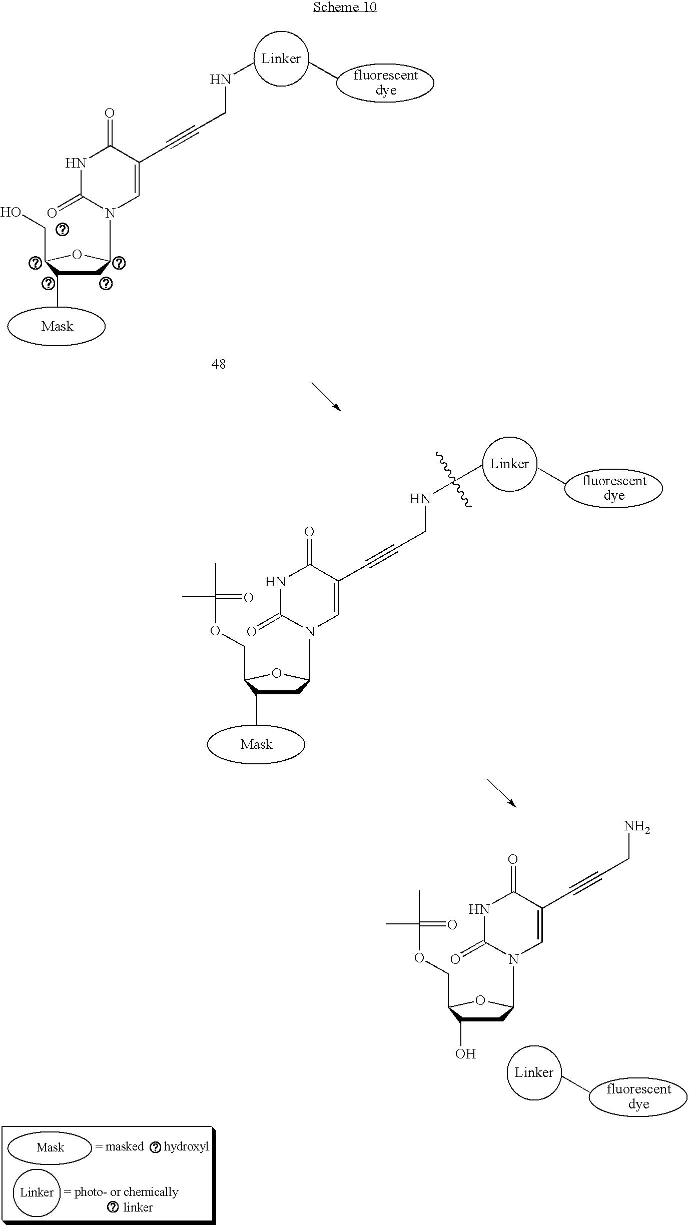

For example, epoxide 49 represents a masked fluorescently labeled DNTP analog (Scheme 11). After incorporation by DNA polymerase, the fluorophore will be cleaved and the epoxide will be opened regioselectively to release the 3′ hydroxyl necessary for subsequent incorporations.

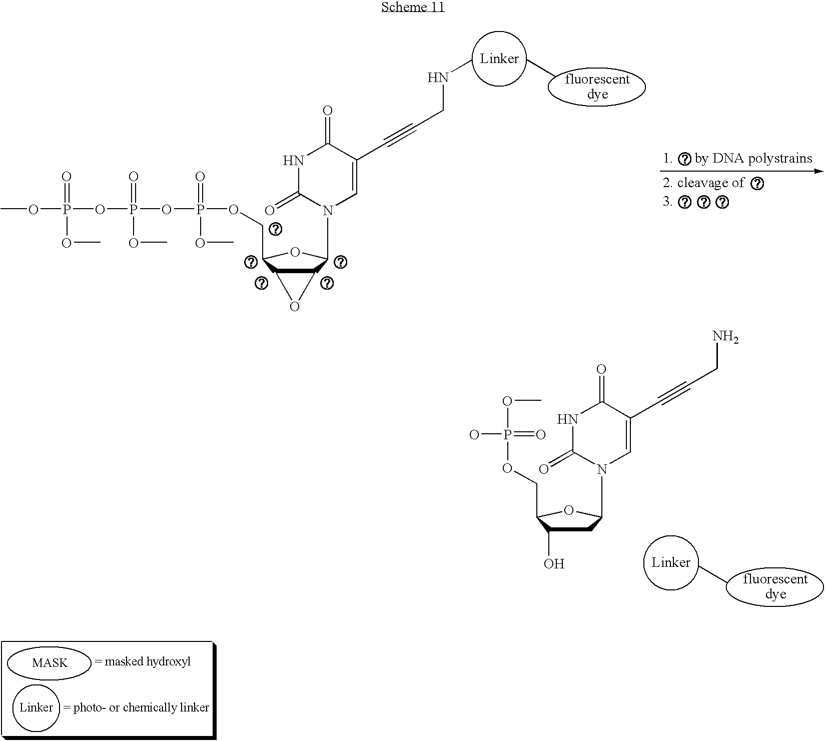

Alternatively, a fluorophore stemming directly from the 3′ position of a fluorescently labeled, chain terminating, nucleoside triphosphates can be synthesized for single-molecule DNA sequencing (50, Scheme 12). After incorporation, cleavage of the fluorophore to liberate the 3′ hydroxyl group occurs. Although uridine is shown as an example, all deoxynucleosides (A,T,U,C,G) are included in this invention.

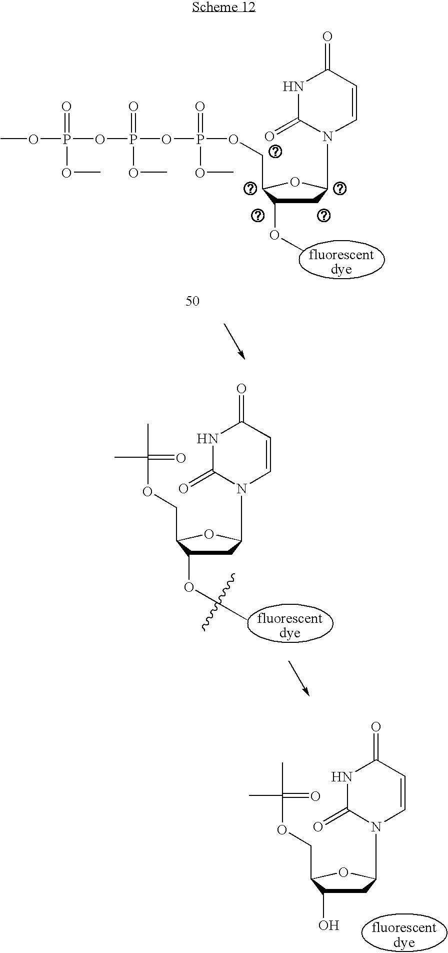

For example, dNTP 51, which contains a chemically cleavable appended fluorophore, can be prepared in a simple one-step procedure from commercially available deoxyuridine triphosphate 50 and succinimidyl ester 33 (Scheme 13).

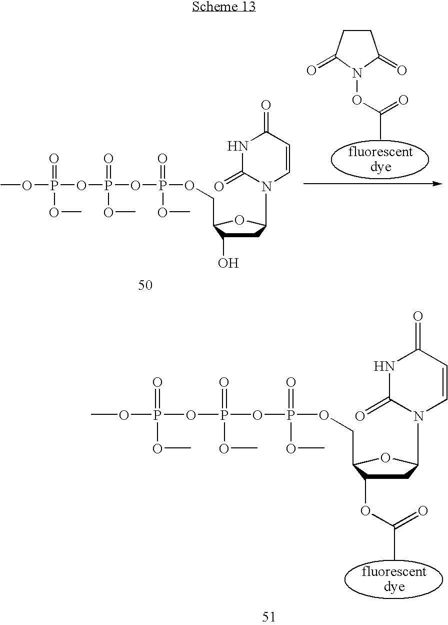

It may be necessary to adopt a more conservative approach to constructing a derivative of 50, as coupling of the secondary alcohol at the 3′ position may be difficult in the presence of the triphosphate group. An alternative, stepwise approach is also shown below (Scheme 14). Silyl protection of the 5′ hydroxyl, esterification of the 3′ alcohol with commercially available acid 53, and liberation of the 5′ hydroxyl will provide 3′ modified 54. This compound can be triphosphorylated and deprotected 55, and then linked to a fluorescent dye 33 to yield the desired ester-bridged labeled dNTP 56.

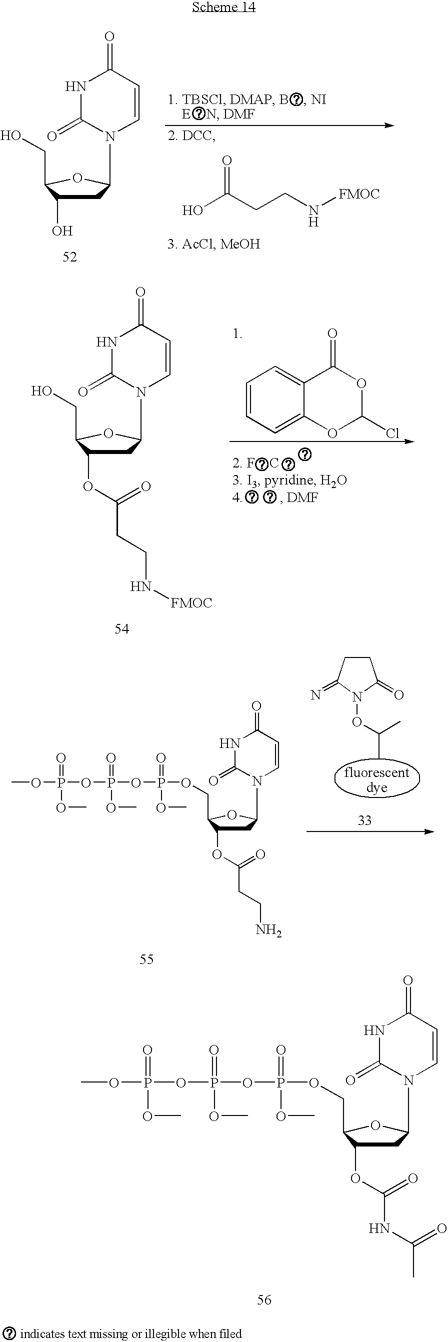

Additional aspects of the invention are described in the following sections and illustrated by the Examples.

Instrument Fabrication

Evanescent wave microscopy (also known as TIR) is an important part of the single-molecule detection scheme. A microscopy set-up with prism-type geometry is not compatible with microfluidic integration. Through-the-objective type TIR (FIG. 8) yields excellent single-molecule sensitivity and allows straightforward integration with microfluidic plumbing. Such systems are available commercially from vendors such as Nikon. A possible design for a microscope system is outlined in FIG. 9. The microscope can be augmented with a computer controlled scanning stage and a temperature controller. Single fluorophore images can be acquired using a state-of-the-art cooled CCD camera.

Directed Evolution of DNA Polymerase

Phage-display based directed evolution is used to engineer novel polymerases capable of incorporating labeled-dNTPs at high efficiency. A schematic of the process is illustrated in FIG. 10.

spFRET Donor Labeled Polymerase

Minimization of background noise is crucial during single-molecule sequencing experiments. spFRET is one way to maximize detection sensitivity while reducing fluorescent noise in single-molecule sequencing experiments. The inherent limitation of the spFRET readout length is approximately 15 by as defined by the Forster radius. This limitation may be overcome by incorporating a new donor-labeled dNTP at regular intervals or by placing the donor on the DNA polymerase. An epitope tag, such as 6-histidine or myc, can be engineered into all DNA polymerase candidates identified through directed evolution. This tag will serve useful at purifying the recombinant polymerase as well as enabling the use of spFRET donor labeled antibodies in the experiments. For example, Cy3 or Europium labeled antibodies can be used as spFRET donors for incorporated Cy5-labeled nucleotides. The labeled antibody will tightly bind the DNA polymerase epitope and may allow for real-time sequence analysis of single molecules. Alternatively, it may be possible to directly label the DNA polymerase with a donor, such as Cy3 or Europium, while retaining enzyme functionality.

Re-Sequencing the Genome of E. coli

To validate the accuracy and fidelity of the single-molecule nucleic acid sequencing technology, the entire genome of E. coli K-12 can be re-sequenced. The genome of this well-known bacteria is already thoroughly sequenced and consists of a singular circular chromosome of approximately 4.64 Mb. A validated protocol can be created using the microfluidic single-molecule nucleic acid sequencing platform to obtain large amounts of shotgun sequence information at a fraction of the cost, time, and manpower required by conventional methodology.

The experiment can be performed on the instrument as described in an earlier section. Images can be directed to a cooled charge-coupled device camera and digitized by a computer. Multiple exposures can be taken of each field of view to compensate for possible intermittency in the fluorophore emission. Custom IDL software can be used to analyze the locations and intensities of fluorescence objects in the intensified charge-coupled device pictures. The resulting traces can be used to determine incorporation information from fluorescently labeled nucleosides triphosphates and deduce the template sequences.

The genomic DNA can be isolated from a fresh culture of E. coli using standard precipitation methods. The isolated genomic DNA can be fragmented by shear force to maximize the randomness of the DNA fragments, followed by treatment with BAL31 nuclease to produce blunt ends. The resulting fragments can be size-fractionated by agarose gel electrophoresis. Fragments between 30 by and 50 by can be excised from the gel and purified.

These fragments can be prepared for anchoring to the surface of the microfluidic chamber by one or both of the following methods. The first method involves ligation of a short double-stranded oligonucleotide, which in the current illustration arbitrarily contains series of A-T base pairs, to the blunt ends of the DNA fragments. Afterwards, successfully ligated fragments can be size fractionated on an agarose gel. The second method requires the enzyme terminal deoxynucleotidetransferase (TDNT), which catalyzes the template independent addition nucleotides to the 3′ end of double-stranded DNA. In the current illustration, incubation of the blunted DNA fragments with TDNT and dATP produces poly(A) 3′ ends. After TDNT treatment, extended fragments can be size fractionated on an agarose gel. Both of these methods can be used as a means to increase coverage of the genome by boosting representation of regions previously found to be difficult or intolerant of subcloning.

The overlap and coverage of these fragments will be sufficient for later assembly of the sequence information. The intact genome of E. coli K-12 is approximately 4.7 million base pairs in length. Approximately 100,000 molecules of double-stranded DNA will represent each genome after fragmentation. In principle, up to 12 million molecules can be resolved on a single 25 mm.times.25 mm surface. Thus, a single sequencing experiment provides coverage of over 100×. According to the Lander and Waterman application of the Poisson distribution, the probability that a base is not sequenced is Pₒ=e^{-m}, where m is the sequence coverage. See, Lander, E. S. et al., Genomic Mapping by Fingerprinting Random Clones: a Mathematical Analysis, Genomics, 2(3): 231-9 (1988). Accordingly, when m=100, Pₒ=3.7×10⁻⁴⁴, an exceedingly low probability that a base will not be sequenced.

The surface of the microfluidic sequencing chamber can be treated as previously described. Surface chemistry based on polyelectrolytes and biotin-streptavidin binding can be used to anchor the DNA fragments to the surface of the microfluidic chamber and to minimize nonspecific binding of dNTPs to the surface. The surface can be immersed alternately in polyallylamine (positively charged) and polyacrylic acid (negatively charged; both from Aldrich) at 2 mg/ml and pH 8 for 10 min each, then washed intensively with distilled water. The carboxyl groups of the last polyacrylic acid layer can serve to prevent the negatively charged labeled dNTPs from binding to the surface of the chamber. In addition, these functional groups can be used for further attachment of a layer of biotin. The chamber surface can be incubated with 5 mM biotin-amine reagent (Biotin-EZ-Link, Pierce) for 10 min in the presence of 1-[3-(dimethylamino)propyl]-3-ethylcarbodiimide hydrochloride (EDC, Sigma) in MES buffer, followed by incubation with Streptavidin Plus (Prozyme, San Leandro, Calif.) at 0.1 mg/ml for 15 min in Tris buffer. Biotinylated, fluorescently labeled sequencing primers can next be deposited onto the streptavidin-coated chamber surface at 10 pM for 10 min in Tris buffer that contains 100 mM MgCl2. In the illustrated example, this primer is an oligo d(T) primer for each of the proposed methods. The prepared DNA fragments can then be denatured and hybridized to the sequencing primers present on the surface of the microfluidic sequencing chamber. At maximum density, 12 million template DNA molecules can be capable of being simultaneously sequenced.

The entire procedure can be automated such that DNA polymerase and labeled dNTPs can be washed in and out of the microfluidic chamber while a CCD camera monitors and records incorporation events on each template DNA molecule. Once the ability to incorporate labeled dNTPs is exhausted, a list of short sequences can be generated using code that has been authored. These short DNA sequences will be suitable for subsequent genome assembly.

Informatics

The front-end image processing part of the data collection can be automated using a set of custom written image analysis routines written in IDL. The software automatically finds feature (i.e. molecule) locations in the images, collects statistics, corrects alignment drifts, and computes sequence statistical information. Software can be written to automate the microfluidic reagent exchange process, to scan the stage, and to create an archive of the raw images and all intermediate calculations. In this manner, the running of the instrument, image acquisition, and conversion of images to sequence data can be automated.

Another aspect of the informatics is to annotate and assemble the short read-length fragments that are obtained in large quantities from each sequencing run. Database software can be developed for the analysis of short transcripts of the yeast and mouse transcriptomes, and this software platform can be used to help analyze the genomic sequence information. It can be merged with the BLAST routine to try to align the fragments against the reference E. Coli genome. De novo assembly (i.e. without using knowledge of the reference E. coli genome) using one of the publicly available sequence assemblers can also be attempted. The difficulty of sequence assembly and re-assembly is directly related to the read-length of the instrument. It is expected that the read-length will be at least 72 base pairs and quite likely substantially greater.

All sequence information can be deposited in public databases such as GenBank. When the instrument is operating reliably in high throughput mode, it can be used as part of a shared facility available to the wider community, and a version can be exported to an NIH genome center.

Gene Expression Analysis Experiments

For gene expression analysis experiments a well characterized cell line can be used, such as NIH_MGC_{—}53 or NIH_MGC_{—}93, for which there is extensive EST and microarray data. See, e.g., Strausberg, R. L., et al., The Mammalian Gene Collection, Science, 286(5439): 455-7 (1999). Validation can be done by comparing the single-molecule results to both conventional microarray data and to data publicly available through the NIH EST database (10,000 clones sequenced).

The experiment can be carried out in much the same way as the re-sequencing of the E. coli genome. Notable differences in procedure are outlined below. Instead of isolating genomic DNA, total RNA can be isolated from NIH_MGC_{—}53 or NIH_MGC_{—}93 cells. One can then proceed in one of the following ways (Scheme 1 or Scheme 2 as shown in FIG. 11) in order to anchor template to the surface of the microfluidic sequencing chamber.

According to Scheme 1 of FIG. 11: Fluorescently labeled biotinylated oligo d(T) primers can be laid onto the PEM/biotin/streptavidin surface as described in the re-sequencing of the E. coli genome section. From the isolated total RNA, poly(A) RNA can be directly hybridized to the oligo d(T) primers present on the sequencing chamber surface. Subsequent sequencing using this technique can be by reverse transcriptase rather than DNA polymerase.

According to Scheme 2 of FIG. 11: Fluorescently labeled biotinylated oligo d(A) oligonucleotides can be bound to the PEM/biotin/streptavidin surface as described in the re-sequencing of the E. coli genome section. From the isolated total RNA, an oligo d(T) primer can be used to synthesize the reverse complement strand from poly(A) RNA using reverse transcriptase. The sample can then be treated with RNAse and the remaining DNA can be laid down onto the oligo d(A) oligonucleotides present on the sequencing chamber surface. Subsequent sequencing using this technique can use random hexamer primers and DNA polymerase.

Experimental Protocols

FRET-Based Method Using Nucleotide-Based Donor Fluorophore

In a first experiment, universal primer is hybridized to a primer attachment site present in support-bound chimeric polynucleotides. Next, a series of incorporation reactions are conducted in which a first fluorescently label nucleoside triphosphate comprising a cyanine-3 donor fluorophore is incorporated into the primer as the first extended nucleotide. If all the chimeric sequences are the same, then a minimum of one fluorescently labeled nucleoside triphosphate must be added as the initial FRET donor because the template nucleotide immediately 3′ of the primer is the same on all chimeric polynucleotides. If different chimeric polynucleotides are used (i.e., the polynucleotide portion added to the bound oligonucleotides is different at least one location), then all four labeled dNTPs initially are cycled. The result is the addition of at least one donor fluorophore to each chimeric strand.

The number of initial incorporations containing the donor fluorophore is limited by either limiting the reaction time (i.e., the time of exposure to donor-labeled nucleoside triphosphates), by polymerase stalling, or both in combination. The inventor has shown that base-addition reactions are regulated by controlling reaction conditions. For example, incorporations can be limited to 1 or 2 at a time by causing polymerase to stall after the addition of a first base. One way in which this is accomplished is by attaching a dye to the first added base that either chemically or sterically interferes with the efficiency of incorporation of a second base. A computer model was constructed using Visual Basic (v. 6.0, Microsoft Corp.) that replicates the stochastic addition of bases in template-dependent nucleic acid synthesis. The model utilizes several variables that are thought to be the most significant factors affecting the rate of base addition. The number of ½ lives until dNTPs are flushed is a measure of the amount of time that a template-dependent system is exposed to dNTPs in solution. The more rapidly dNTPs are removed from the template, the lower will be the incorporation rate. The number of wash cycles affects the number of bases ultimately added to the extending primer. The number of strands to be analyzed is a variable of significance when there is not an excess of dNTPs in the reaction. Finally, the slowdown rate is an approximation of the extent of base addition inhibition, usually due to polymerase stalling.

The model demonstrates that, by controlling reaction conditions, one can precisely control the number of bases that are added to an extending primer in any given cycle of incorporation. At a constant rate of inhibition of second base incorporation (i.e., the inhibitory effect of incorporation of a second base given the presence of a first base), the amount of time that dNTPs are exposed to template in the presence of polymerase determines the number of bases that are statistically likely to be incorporated in any given cycle (a cycle being defined as one round of exposure of template to dNTPs and washing of unbound dNTP from the reaction mixture). When time of exposure to dNTPs is limited, the statistical likelihood of incorporation of more than two bases is essentially zero, and the likelihood of incorporation of two bases in a row in the same cycle is very low. If the time of exposure is increased, the likelihood of incorporation of multiple bases in any given cycle is much higher. Thus, the model reflects biological reality. At a constant rate of polymerase inhibition (assuming that complete stalling is avoided), the time of exposure of a template to dNTPs for incorporation is a significant factor in determining the number of bases that will be incorporated in succession in any cycle. Similarly, if time of exposure is held constant, the amount of polymerase stalling will have a predominant effect on the number of successive bases that are incorporated in any given cycle. Thus, it is possible at any point in the sequencing process to add or renew donor fluorophore by simply limiting the statistical likelihood of incorporation of more than one base in a cycle in which the donor fluorophore is added.

Upon introduction of a donor fluorophore into the extending primer sequence, further nucleoside triphosphates comprising acceptor fluorophores (here, cyanine-5) are added in a template-dependent manner. It is known that the Foster radius of Cy-3/Cy5 fluorophore pairs is about 5 nm (or about 15 nucleotides, on average). Thus, donor must be refreshed about every 15 bases. This is accomplished under the parameters outlined above. In general, each cycle preferably is regulated to allow incorporation of 1 or 2, but never 3 bases. So, refreshing the donor means simply the addition of all four possible nucleotides in a mixed-sequence population using the donor fluorophore instead of the acceptor fluorophore every approximately 15 bases (or cycles).

The methods described above are alternatively conducted with the FRET donor attached to the polymerase molecule. In that embodiment, donor follows the extending primer as new nucleoside triphosphates bearing acceptor fluorophores are added. Thus, there typically is no requirement to refresh the donor. In another embodiment, the same methods are carried out using a nucleotide binding protein (e.g., DNA binding protein) as the carrier of a donor fluorophore. In that embodiment, the DNA binding protein is spaced at intervals (e.g., about 5 nm or less) to allow FRET. Thus, there are many alternatives for using FRET to conduct single molecule sequencing using the devices and methods taught in the application. However, it is not required that FRET be used as the detection method. Rather, because of the intensities of the FRET signal with respect to background, FRET is an alternative for use when background radiation is relatively high.

Non-FRET Based Methods

Methods for detecting single molecule incorporation without FRET are also conducted. In this embodiment, incorporated fluorescently labeled nucleoside triphosphates are detected by virtue of their optical emissions after sample washing. Primers are hybridized to the primer attachment site of bound chimeric polynucleotides. Reactions are conducted in a solution comprising Klenow fragment Exo-minus polymerase (New England Biolabs) at 10 nM (100 units/ml) and a labeled nucleoside triphosphate in EcoPol reaction buffer (New England Biolabs). Sequencing reactions takes place in a stepwise fashion. First, 0.2 .mu.M dUTP-Cy3 and polymerase are introduced to support-bound chimeric polynucleotides, incubated for 6 to 15 minutes, and washed out. Images of the surface are then analyzed for primer-incorporated U-Cy5. Typically, eight exposures of 0.5 seconds each are taken in each field of view in order to compensate for possible intermittency (e.g., blinking) in fluorophore emission. Software is employed to analyze the locations and intensities of fluorescence objects in the intensified charge-coupled device pictures. Fluorescent images acquired in the WinView32 interface (Roper Scientific, Princeton, N.J.) are analyzed using ImagePro Plus software (Media Cybernetics, Silver Springs, Md.). Essentially, the software is programmed to perform spot-finding in a predefined image field using user-defined size and intensity filters. The program then assigns grid coordinates to each identified spot, and normalizes the intensity of spot fluorescence with respect to background across multiple image frames. From those data, specific incorporated nucleoside triphosphate are identified. Generally, the type of image analysis software employed to analyze fluorescent images is immaterial as long as it is capable of being programmed to discriminate a desired signal over background. The programming of commercial software packages for specific image analysis tasks is known to those of ordinary skill in the art. If U-Cy5 is not incorporated, the substrate is washed, and the process is repeated with dGTP-Cy5, dATP-Cy5, and dCTP-Cy5 until incorporation is observed. The label attached to any incorporated nucleoside triphosphate is neutralized, and the process is repeated. To reduce bleaching of the fluorescence dyes, an oxygen scavenging system can be used during all green illumination periods, with the exception of the bleaching of the primer tag.

In order to determine a template sequence, the above protocol is performed sequentially in the presence of a single species of labeled dATP, dGTP, dCTP or dUTP. By so doing, a first sequence can be compiled that is based upon the sequential incorporation of the nucleotides into the extended primer. The first compiled sequence is representative of the complement of the chimeric polynucleotide. As such, the sequence of the chimeric polynucleotides can be easily determined by compiling a second sequence that is complementary to the first sequence. Because the sequence of the oligonucleotide is known, those nucleotides can be excluded from the second sequence to produce a resultant sequence that is representative of the target nucleic acid.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

1-25. (canceled)

26. A method for analyzing a ribonucleic acid sequence, the method comprising:
contacting an RNA molecule with a primer to generate a primed template;
exposing four types of nucleotides to the primed template, wherein at least one of said types of nucleotides comprises a detectable label;
exposing the mixture of nucleotides and the primed template to a transcriptase or polymerase;
permitting incorporation of a labeled nucleotide onto the primer; and
detecting said incorporation, thereby analyzing the ribonucleic acid sequence.

27. The method of claim 26, wherein the primer, nucleotides and polymerase are present in a single buffer.

28. The method of claim 26, wherein the primer, nucleotides and polymerase are added independently or in various combinations thereof.

29. The method of claim 26, wherein the detecting step is carried out at a rate as fast or faster than the rate at which said labeled nucleotide is added onto the primer.

30. The method of claim 26, wherein the detecting step is carried out by imaging the labeled nucleotide upon incorporation.

31. The method of claim 26, wherein the primer comprises a nucleotide sequence homologous in whole or in part to the known nucleotide sequence of a gene.

32. The method of claim 26, wherein the primer comprises an oligo dT.

33. The method of claim 26, wherein the primer comprises a set of random primers.

34. The method of claim 26, wherein the detecting is carried out for about 15 to 100 cycles to obtain sequence information associated with 15-100 base pairs of the ribonucleic acid.

35. The method of claim 26, wherein the detecting is carried out for about 20-30 cycles to obtain sequence information associated with 20-30 base pairs of the ribonucleic acid.

36. The method of claim 26, wherein the primer or template is biotinylated.

37. The method of claim 26, further comprising attaching the primer or template to a substrate.

38. The method of claim 33, wherein the primer or template is attached to a substrate with streptavidin.

39. The method of claim 26, further comprising comparing the ribonucleotide sequence information to a sequence database.

40. The method of claim 26, wherein the transcriptase is a reverse transcriptase.

41. The method of claim 26, wherein the ribonucleotide nucleic acid sequence comprises cDNA sequence information.

42. The method of claim 26, wherein the RNA molecule comprises mRNA.

43. The method of claim 42, wherein the mRNA is obtained from total cellular RNA.

44. A method for analyzing a ribonucleic acid sequence, the method comprising:
contacting an RNA molecule with a primer to generate a primed template;
reverse transcribing the RNA molecule in the presence of four types of nucleotides and a reverse transcriptase to generate a minus strand cDNA;
exposing four types of nucleotides to the minus strand cDNA in the presences of primer described in claim 31, wherein at least one of said types of nucleotides comprises a detectable label;
exposing the mixture of nucleotides and the primed template to a polymerase;
permitting incorporation of a labeled nucleotide onto the primer; and detecting said incorporation in real time, thereby analyzing the ribonucleic acid sequence.

45. The method of claim 26 or 44, further comprising cleaving the detectably labeled nucleotide.

46. The method of claim 45, wherein the cleavage is performed by using photolysis or chemical hydrolysis.

47. The method of claim 26 or 44, wherein the detectable label is selected from the group consisting of cyanine, rhodamine, fluorescein, coumarin, BODIPY, alexa, and conjugated multi-dyes.

48. The method of claim 44, wherein the RNA is attached to a substrate.

49. The method of claim 47, wherein the cDNA is isolated prior to contact with the detectably labeled nucleotides.

50. A method for analyzing a ribonucleic acid sequence, the method comprising:
contacting an RNA molecule with a primer to generate a primed template;
reverse transcribing the RNA molecule in the presence of four types of nucleotides and a reverse transcriptase to generate a minus strand cDNA;
exposing four types of nucleotides to the minus strand cDNA in the presences of primer described in claim 32, wherein at least one of said types of nucleotides comprises a detectable label;
exposing the mixture of nucleotides and the primed template to a polymerase;
permitting incorporation of a labeled nucleotide onto the primer; and detecting said incorporation in real time, thereby analyzing the ribonucleic acid sequence.

51. The method of claim 50, further comprising cleaving the detectably labeled nucleotide.

52. The method of claim 51, wherein the cleavage is performed by using photolysis or chemical hydrolysis.

53. The method of claim 50, wherein the detectable label is selected from the group consisting of cyanine, rhodamine, fluorescein, coumarin, BODIPY, alexa, and conjugated multi-dyes.

54. The method of claim 50, wherein the RNA is attached to a substrate.

55. The method of claim 53, wherein the cDNA is isolated prior to contact with the detectably labeled nucleotides.
